(19) United States
(12) Patent Application Publication
(10) Pub. No.: US 2024/0001156 A1
(43) Pub. Date: Jan. 4, 2024

(54) APPARATUS, METHOD, AND USE FOR ULTRASOUND MEDIATED MICROBUBBLE DELIVERY OF PHARMACEUTICAL COMPOSITIONS

(71) Applicants: Warren LEE, Toronto (CA); Howard LEONG-POI, Toronto (CA); Raffi KARSHAFIAN, Thorhill (CA)

(72) Inventors: Warren LEE, Toronto (CA); Howard LEONG-POI, Toronto (CA); Raffi KARSHAFIAN, Thorhill (CA)

(21) Appl. No.: 18/368,248

(22) Filed: Sep. 14, 2023

Related U.S. Application Data
(63) Continuation of application No. 16/475,710, filed on Jul. 3, 2019, now abandoned.
(60) Provisional application No. 62/442,709, filed on Jan. 5, 2017.

(51) Int. Cl. A61N 7/00; A61K 47/69; A61K 9/00; A61K 31/7105; C12N 15/113

(52) U.S. Cl. CPC A61N 7/00; A61K 47/6925; A61K 9/0019; A61K 31/7105; C12N 15/113; A61K 45/06

(57) ABSTRACT

An apparatus, method, and use for ultrasound mediated microbubble delivery of pharmaceutical compositions to pulmonary tissue are provided. The pulmonary ultrasound apparatus includes an ultrasound signal generator for generating ultrasonic signals, an ultrasound transducer assembly having an ultrasound transducer operatively connected to the ultrasound signal generator, the ultrasound transducer configured to transmit the ultrasound signal generated by the ultrasound signal generator to pulmonary tissue, wherein the ultrasonic signal is transmitted at a frequency, a pressure, and a pulse duration for cavitating microbubbles to deliver a pharmaceutically active molecule to the pulmonary tissue.

### RELATED APPLICATIONS

This non-provisional application claims priority from U.S. provisional application No. 62/442,709 filed on Jan. 5, 2017, which is incorporated herein by reference in its entirety.

### FIELD OF THE APPLICATION

The disclosure relates to apparatus, methods, and uses for ultrasound mediated microbubble delivery of pharmaceutical compositions for treating pulmonary edema.

### BACKGROUND

Pulmonary edema is defined as any condition characterized by fluid accumulation in the tissue and air spaces of the lungs. Conditions and diseases that can cause pulmonary edema include acute respiratory distress syndrome (ARDS), chronic obstructive pulmonary disease (COPD), cystic fibrosis (CF), congestive heart failure, and pneumonia, among others.

Acute respiratory distress syndrome (ARDS) is a devastating disorder characterized by pulmonary edema leading to severe hypoxemia and diminished lung compliance. In ARDS the smallest blood vessels in the lung exhibit increased permeability, allowing for the leakage of protein-rich fluid into the airspaces, causing patients to essentially drown on their own fluids. The causes of ARDS are myriad but are dominated by infections, the most common causes being bacterial and viral pneumonia, followed by diverse pulmonary and extrapulmonary insults including intra-abdominal sepsis, aspiration and severe trauma. Regardless of the cause however, CT scans of the lungs of ARDS patients characteristically reveal heterogeneous disease, with areas of injured lung interspersed with relatively normal-appearing regions. This heterogeneity greatly complicates disease management. Mechanical ventilation, although life-saving, preferentially inflates the normal regions of lung, causing over-distension and lung damage. Analogously, pharmaceutical agents administered by inhalation preferentially distribute to the most normal regions of the lung, potentially causing adverse or off-target effects. A similar limitation applies to drugs administered systemically, which distribute to organs throughout the body and not just to the injured lung, thereby predisposing the patient to adverse effects. This inability to target just the injured areas of the lung in ARDS remains one of the thorniest problems in critical care.

The fundamental defect in ARDS is leakage of the alveolar-capillary membrane, a thin and delicate structure composed of a single endothelial monolayer abutting the alveolar epithelium, separated only by strands of connective tissue. Leakage of this barrier is caused by a combination of epithelial damage, endothelial permeability and impaired fluid clearance. There is now growing recognition that the loss of endothelial barrier integrity in combination with excessive endothelial activation plays a critical role in determining mortality. The endothelium lining in the pulmonary capillaries is continuous (i.e. no gaps between cells) and the development of inter-endothelial discontinuity can induce alveolar edema. While epithelial damage also contributes to lung injury, some data suggest that the loss of endothelial barrier integrity is the sine qua non to the formation of pulmonary edema in ARDS. Indeed, it has been reported that apoptosis of the alveolar epithelium alone is not sufficient to induce lung vascular leak. ARDS is sadly common in critical care units and the mortality rate is as high as 40% and accounts for almost 25% of patients on mechanical ventilators. Thus, strategies to enhance endothelial barrier function or endothelial repair are critically important.

In the case of ARDS caused by bacterial pneumonia, treatment requires a two-pronged approach: first, the administration of antibiotics to kill the bacterial pathogen and second, non-specific supportive care (e.g. lung-protective mechanical ventilation) to allow the injured lungs time to heal. However, in this era of pathogen resistance and dwindling new antimicrobial development, even the provision of appropriate antimicrobial therapy has become increasingly problematic. While enteric gram-negative bacilli make up the plurality of isolates responsible for ventilator-associated pneumonia, antibiotic resistance is becoming ever-prevalent. Gram-negative isolates remain sensitive to aminoglycosides such as tobramycin and gentamicin, however the use of this class of antibiotics for pneumonia has been discouraged because of poor lung penetration and because even therapeutic levels are associated with nephrotoxicity and ototoxicity. While inhaled, tobramycin has been used to decrease the bacterial burden in the airways, however its efficacy in pneumonia may be limited because the inhaled medication is preferentially deposited in the uninjured (and less infected) regions of the lung. Analogous considerations apply to methicillin-resistant Staphylococcus aureus (MRSA), a major gram-positive cause of hospital-acquired pneumonia. While the antibiotic vancomycin is routinely prescribed for MRSA pneumonia, its penetration into lung tissue is not very high. Thus, developing a practical and safe method to increase the therapeutic index of aminoglycosides and vancomycin specifically in the lungs would be of great clinical value.

Microbubbles are made up of a gas-filled core and an outer lipid, protein or polymer shell and are generally smaller than ten micrometres in diameter. Microbubbles are used routinely for echocardiographic studies and have widespread application in industry, life science, and medicine. Microbubbles are also routinely used as contrast agents in diagnostic ultrasound and have been shown to enhance the delivery of pharmaceutical products such as drugs or genes to tissues. When microbubbles are placed in an ultrasound field, they undergo stable and inertial cavitation. This cavitation induces shear stress on biological membranes within their vicinity, leading to the formation of transient pores in the plasma membranes of cells and enhanced endocytosis, thereby allowing uptake of local pharmaceutical products such as drugs or genes.

Wei J. Cao, Joshua D. Rosenblat, Nathan C. Roth, Michael A. Kuliszewski, Pratiek N. Matkar, Dmitriy Rudenko, Christine Liao, Paul J. H. Lee, Howard Leong-Poi. Therapeutic Angiogenesis by Ultrasound-Mediated MicroRNA-126-3p Delivery. Arterioscler Thromb Vase Biol. 2015; 35:2401-241 1. DOL10.1 161/ATVBAHA. 115.306506, discloses ultrasound-targeted microbubble destruction as a non-invasive method of targeted gene delivery using ultrasonic destruction of intravenously administered DNA-bearing microbubbles.

### SUMMARY

In other parts of the body, ultrasound waves have been used to deliver restorative genes and medicines to diseased tissues. However, the medical and scientific community has long thought that ultrasound administration to the lung is impossible because air blocks ultrasound waves. It has now been found that because the injured lung is filled with fluid and not air, ultrasound waves can penetrate the injured regions of the lung while leaving the normal lung untouched. Ultrasound waves can therefore be used to deliver genes and drugs just to the injured lung, sparing other organs in the body and sparing the normal air-filled regions of the lung.

Ultrasound of the lung is difficult since the air in the lungs causes reflection and scattering of ultrasound energy. Ultrasound is typically transmitted via a medium such as a gel or a fluid.

However, since ARDS symptoms include fluid-filled areas of injured lung, the qualities of ultrasound can be harnessed to treat ARDS. That is, ultrasound waves can be used to treat injured areas of the lung (since they are filled with fluid) while leaving the normal, air-filled areas of the lung unaffected.

Furthermore, by targeting the ultrasound probe to the chest, the ultrasound energy is focused on the thorax thereby preventing an effect on other organs. Using this non-invasive approach, ultrasound and microbubble-induced drug or gene delivery is targeted not just to the lungs but specifically to the injured areas of the lung, providing directed medicine to the most critically ill patients.

What is provided in an aspect is:

Use of ultrasound-mediated microbubble pharmaceutical product delivery to treat pulmonary edema;

Use of ultrasound-mediated microbubble pharmaceutical product delivery to treat ARDS;

Lung-specific ultrasound transducers that may accomplish the following:

Targeting of injured lung, with minimal or no penetration of normal lung areas; the emitters of the chest-transducer may fit between patients' ribs and be adjustable for each patient; minimal or no movement of the patient may be required;

Avoidance of heart and other non-pulmonary tissue to ultrasound exposure; and

Incorporation of a treatment algorithm that may optimize microbubble cavitation and minimize unnecessary ultrasound exposure (i.e. the beam turns off when microbubbles are no longer detected)

Given the documented safety of ultrasound in ICU patients, repeated treatments are envisioned.

To apply ultrasound waves to areas of the lung that are deep within the chest, described herein is a pulmonary ultrasound apparatus, and an endobronchial ultrasound transducer.

Also provided, in another aspect, is a pulmonary ultrasound apparatus comprising, an ultrasound signal generator for generating an ultrasonic signal, an ultrasound transducer assembly having an ultrasound transducer operatively connected to the ultrasound signal generator, the ultrasound transducer configured to transmit the ultrasound signal generated by the ultrasound signal generator to pulmonary tissue, wherein the ultrasonic signal is transmitted at a frequency, a pressure, and a pulse duration for cavitating microbubbles to deliver a pharmaceutically active molecule to the pulmonary tissue.

In an embodiment, the ultrasound transducer assembly has a flexible planar body for covering the at least part of the chest, and without movement of the ultrasound transducer assembly, the ultrasound transducer transmits the ultrasonic signal to the pulmonary tissue. In another embodiment, the ultrasound transducer has a width and a length about the size of the intercostal muscles for transmitting the ultrasonic signal between the ribs. In another embodiment, the ultrasound transducer assembly has an elongated body for endobronchial insertion, and the ultrasound transducer is at a distal end of the elongated body for transmitting the ultrasonic signal to the pulmonary tissue.

In another embodiment, the ultrasound transducer directs targeted ultrasound signals to cavitate the microbubbles only when the microbubbles are detected in the pulmonary tissue. In another embodiment, the ultrasound transducer assembly has a movement mechanism for moving the ultrasound transducer assembly within the ultrasound transducer assembly. Another embodiment further comprises, an array of ultrasound transducers, the array of ultrasound transducer spaced apart a distance and each ultrasound transducer is operatively connected to the ultrasound signal generator. Another embodiment further comprises, a 2-dimensional array of ultrasound transducers, the array of ultrasound transducer spaced apart a first distance in a first dimension and a second distance in a second dimension, and each ultrasound transducer is operatively connected to the ultrasound signal generator. In another embodiment, the ultrasound transducer or the array of ultrasound transducers is capable of imaging, treating, or both imaging and treating an entire lung or both lungs entirely. In another embodiment, the ultrasound transducer assembly is configured to maintain an adjustable spacing between the ultrasound transducers.

Also provided, in another aspect, is an intravenous composition for treating pulmonary edema, the composition comprising, microbubbles, a pharmaceutically active molecule and a pharmaceutically acceptable carrier.

In an embodiment, the pharmaceutically active molecule is at least one of microRNA, antagomir, and blockmir. In another embodiment, the microRNA is at least one of miRNA-126, miRNA-150, miRNA-181b, miRNA126-3p, and miRNA150-5p; the antagomir is at least one of an antagomir to miRNA-27A and miRNA-146b; and the blockmir is at least one of a blockmir to miRNA-27A and miRNA-146b. In another embodiment, the pharmaceutically active molecule is at least one of an aminoglycoside, steroid, antibiotic, vancomycin, antiviral, endothelial barrier-enhancing drug, and keratinocyte growth factor (KGF). In another embodiment, the endothelial barrier-enhancing drug is one or more of vasculotide, adrenomedullin, fasudil, imatinib, atrial natriuretic peptide, and SIP. Another embodiment comprises, microRNA and at least one of an antibiotic compound and an antiviral compound. In another embodiment, the microbubble is coformulated with the pharmaceutically active compound. In another embodiment, the microbubble is formulated independently and added to a solution of pharmaceutically active compound. In another embodiment, the microbubble is bound to the pharmaceutically active compound.

Also provided, in another aspect, is a use of pulmonary ultrasound to treat pulmonary edema comprising, providing an intravenous composition to a patient comprising a plurality of microbubbles, a pharmaceutically active compound, and a pharmaceutically acceptable carrier, and applying ultrasound to the patient at a target of pulmonary edema to cavitate the microbubbles and deliver the pharmaceutically active compound to the patient.

In an embodiment, the pharmaceutically active molecule is at least one of a microRNA, antagomir, and blockmir. In another embodiment, the microRNA is one or more of miRNA-126, miRNA-150, miRNA-181b, miRNA126-3p, and miRNA150-5p; the antagomir is at least one of an antagomir to miRNA-27A and miRNA-146b; and the blockmir is at least one of a blockmir to miRNA-27A and miRNA-146b. In another embodiment, the pharmaceutically active molecule is at least one of an aminoglycoside, antibiotic, steroid, vancomycin, antiviral, endothelial barrier-enhancing drug, and keratinocyte growth factor (KGF). In another embodiment, the endothelial barrier-enhancing drug is one or more of vasculotide, adrenomedullin, fasudil, imatinib, atrial natriuretic peptide, and SIP. In another embodiment, the intravenous composition comprises microRNA and at least one of an antibiotic compound and an antiviral compound.

Also provided, in another aspect, is a method of treating pulmonary edema, the method comprising, administering intravenously to a patient microbubbles, and a pharmaceutically active molecule, and irradiating the patient with ultrasound at a target of pulmonary edema to deliver the pharmaceutically active compound to the patient.

In an embodiment, the pulmonary edema is associated with acute respiratory distress syndrome. In another embodiment, the pulmonary edema is associated with cystic fibrosis. In another embodiment, the pulmonary edema is associated with congestive heart failure. In another embodiment, the pharmaceutically active molecule is one or more of microRNA, an aminoglycoside, vancomycin, antiviral, endothelial barrier-enhancing drug, and keratinocyte growth factor (KGF). In another embodiment, the microbubbles and the pharmaceutically active molecule are delivered to the patient simultaneously. In another embodiment, the microbubbles and the pharmaceutically active molecule are delivered to the patient sequentially. In another embodiment, the target of pulmonary edema is lung endothelium lining.

Also provided, in another aspect, is a method for delivering a pharmaceutical active molecule to a site of pulmonary edema, comprising, introducing microbubbles to an area proximate to the site of pulmonary edema, and directing an ultrasonic signal to the site of pulmonary edema, the ultrasonic signal for cavitating the microbubbles to deliver the pharmaceutically active molecule to the site of pulmonary edema.

An embodiment further comprises, scanning a chest cavity to identify internal structures including one or more organs, site of pulmonary edema, injured lung tissue, and healthy lung tissue. Another embodiment further comprises, scanning the site of pulmonary edema for echogenicity changes in the injured lung tissue after delivery of the pharmaceutically active molecule to the site of pulmonary edema. Another embodiment further comprises, detecting whether microbubbles are in the area proximate to the site of pulmonary edema. Another embodiment further comprises, directing an ultrasonic signal to the site of pulmonary edema, the ultrasonic signal for cavitating the microbubbles, only when the microbubbles are detected in the area proximate to the site of pulmonary edema.

### BRIEF DESCRIPTION OF THE FIGURES

FIG. 1 (SHEET 1/26) depicts a block diagram of an example embodiment of the apparatus.

FIG. 2 (SHEET 2/26) depicts a block diagram of an alternate embodiment of the apparatus.

FIG. 3 (SHEET 3/26) depicts a block diagram of an alternate embodiment of the apparatus.

FIG. 4A (SHEET 4/26) depicts a block diagram of an alternate embodiment of the apparatus for endobronchial use.

FIG. 4B (SHEET 5/26) depicts a cross-sectional diagram of an embodiment of the apparatus of FIG. 4A.

FIG. 5 (SHEET 6/26) depicts a block diagram of an example embodiment of the method.

FIG. 6A-FIG. 6D (SHEET 7/26 and SHEET 8/26) depict a method for treating a fluid-filled portion of an injured lung.

FIG. 7A (SHEET 9/26) depicts a representation of a fluid-filled portion of an injured lung of FIG. 6C.

FIG. 7B (SHEET 10/26) depicts an alternate representation of a fluid-filled portion of an injured lung of FIG. 6C.

FIG. 8 (SHEET 11/26) depicts a flow diagram that illustrates an embodiment of the method.

FIG. 9 (SHEET 12/26) depicts a flow diagram that illustrates an alternate embodiment of the method.

FIG. 10 (SHEET 13/26) depicts a flow diagram that illustrates an alternate embodiment of the method.

FIG. 11 (SHEET 14/26) illustrates an ultrasound mediated pulmonary drug delivery approach using microbubbles.

FIG. 12 (SHEET 15/26) is a graph illustrating pulmonary oxygen saturation in the presence and absence of microbubble ultrasound treatment.

FIG. 13 (SHEET 16/26) is a set of histopathological images of lung sections of mice.

FIGS. 14A and 14B (SHEET 17/26) are graphs of two experiments determining the effect of ultrasound-microbubble treatment on the efficacy of gentamicin for E. coli pneumonia in mice.

FIG. 15 (SHEET 18/26) is a graph showing USMB enhanced deposition of miRNA in edematous mouse lung.

FIG. 16A (SHEET 19/26) is a gel showing miRNA activity on primary human lung microvascular endothelial cells infected with influenza A.

FIGS. 16B and 16C (SHEET 19/26) are histopathological images of lung sections (hematoxylin and eosin stain) from C57BL/6 mice infected with human influenza A.

FIG. 17 (SHEET 20/26) depicts a perspective view of another embodiment of the apparatus in relation to the top and the bottom of a patient's chest, with the patient lying on a hospital bed.

FIG. 18 (SHEET 21/26) depicts an overhead view of another embodiment of the apparatus in relation to a patient on a hospital bed.

FIG. 19 (SHEET 22/26) depicts an overhead view of the embodiment of the apparatus shown in FIG. 18 in relation to a patient's chest.

FIG. 20 (SHEET 23/26) depicts a side view of another embodiment of the apparatus in relation to the top and the bottom of a patient's chest, with the patient lying on a hospital bed.

FIG. 21 (SHEET 24/26) depicts a sectional view of the embodiment of the apparatus shown in FIG. 20 in relation to the top and bottom of a patient's chest.

FIG. 22 (SHEET 25/26) depicts a perspective view of the embodiment of the apparatus shown in FIG. 21.

FIG. 23 (SHEET 26/26) depicts a simplified perspective view of the embodiment of the apparatus shown in FIG. 22.

### LISTING OF REFERENCE NUMERALS USED IN THE DRAWINGS

100—Apparatus
102—Ultrasound Signal Generator
104—Ultrasound Transducer Assembly
106—Ultrasound Transducer
108—Patient
109—Bed
200—Lung
201—Rib
202—Ultrasonic signal
203—Heart
204—Injured Lung
300—Fluid
302—Bronchial Capillary
304—Alveolus
306—Microbubbles
400—Pharmaceutical product/Pharmaceutically active compound
500—Patient Chest
502—Patient Back
504—Esophagus
506—Right Bronchi
508—Left Bronchi
600—apply step
602—post scan step
700—pre-scan step
702—injured lung decision step
704—move/activate transducer step
800—detect microbubbles step

### DETAILED DESCRIPTION OF THE NON-LIMITING EMBODIMENTS

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the invention is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Field, Background, Summary or the following Detailed Description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described.

The application of microbubbles in combination with an ultrasound apparatus for the treatment of pulmonary edema takes advantage of the fact that while ultrasound beams cannot penetrate air and hence will be deflected by normal lung, ultrasound beams will penetrate fluid-filled or collapsed (i.e. injured) lung. By directing an ultrasound apparatus at injured, diseased, or fluid-filled lung, drugs delivered concurrently with microbubbles can be administered selectively to the injured regions of the lung and provide directed treatment to enhance the killing of bacterial pathogens in the lung. Specifically, the presently described apparatus, composition, use and method can be used to treat pulmonary edema, and specifically ARDS, with microbubbles to deliver pharmaceutically active molecules such as, for example, antibiotics, micro-RNA (miRNA), antivirals, steroids, and other chemotherapeutics. In particular, targeted miRNA delivery to injured regions of the lung can support repair of the endothelial barrier and attenuate lung injury.

The presently described pulmonary ultrasound apparatus provides a customized ultrasound transducer taking into consideration the presence of the ribs (bone) such that the ultrasound waves can be controlled to provide a treatment planning algorithm to expose only the lungs to pulses. Accordingly, by the present design ultrasound waves can be selectively directed at the lungs, and at particular injured locations in the lungs and thorax, while avoiding the heart and other organs.

The combination of pulmonary ultrasound and microbubbles enables only the injured locations in the lung to receive targeted treatment, limiting systemic patient exposure to toxic chemotherapeutics and delivering active drug to the required location. In this way, the present apparatus, composition, use, and method can provide targeted delivery of molecules preferentially to the injured regions of the lung. From a therapeutic perspective, it has been found that endothelial permeability to fluid and to leukocytes (e.g. diapedesis) can be regulated separately, and that inhibition of pulmonary edema can be accomplished without compromising alveolar neutrophil recruitment, which is a key component of the innate immune response to an inciting pathogen.

Endothelial permeability during inflammation involves the formation of gaps between adjacent cells. These occur due to the remodeling or dismantling of the cell-cell junctions, of which tight junctions and adherens junctions are the major players in maintaining barrier function. Tight junction strands form a physical barrier preventing the passage of solutes between cells and are composed of numerous proteins including claudins, occludins, junctional adhesion molecules, and ZO proteins. Of these, claudins and occludins dominate. In contrast, in adherens junctions the major constituent is VE-cadherin. Cytokines and other mediators can induce the internalization and/or degradation of plasmalemmal VE-cadherin, or can alter its association with other proteins (e.g. p120-catenin, β-catenin) by phosphorylation. These perturbations of VE-cadherin are sufficient to increase endothelial permeability. Furthermore, upregulation of VE-cadherin or its selective targeting to the plasmalemma is barrier-protective.

Another major regulator of the endothelial barrier is the Tie2-angiopoietin axis. Tie2 is a transmembrane receptor tyrosine kinase that is highly enriched in endothelial cells; its engagement by angiopoietin-1 (Ang1) leads to enhanced endothelial survival, decreased endothelial NFKB activation and enhanced barrier stability via effects on VE-cadherin and the actin cytoskeleton. In contrast, angiopoietin-2 (Ang2) acts as a competitive inhibitor to Ang1, impeding Ang1 signaling via Tie2 and inducing endothelial leakage. Inflammatory diseases and severe infections are characterized by increased Ang2 levels, decreased Ang1 levels and loss of Tie2 expression. In addition to changes at the intercellular junctions, endothelial apoptosis or damage can create gaps in the monolayer. There are numerous additional putative causes of lung edema, such as neutrophil recruitment and activation, alveolar epithelial injury and a role for platelets. Instead, the recent realization that vascular permeability to fluids can be separated from leukocyte diapedesis is the impetus for the focus on the endothelium.

To provide ultrasound therapy to patients with pulmonary edema, ultrasound can be applied by the present apparatus to the surface of the chest. Air-filled regions of lung cause scattering or blocking of the ultrasound waves. In contrast, damaged areas of a lung with ARDS are filled with fluid or are atelectatic (completely or partially collapsed) and allow penetration of the ultrasound beam. Cavitation of bloodstream circulating microbubbles caused by the ultrasound pulse leads to enhanced cellular uptake of drugs or pharmaceutically active molecules in the vicinity of the microbubbles. The effect is similar whether drugs are bound to or encapsulated by the microbubble, bound to the microbubble, or merely in close proximity to the microbubble.

In an embodiment, the microbubbles may be formed from liquid droplets (e.g. perfluorocarbon) through either phase-transition or cavitation. The microbubbles are generally 2-5 times larger than the liquid droplets. A skilled person would understand this to be the method of acoustic droplet vaporization (ADV). The half-life of droplets in tissue is in the range of days. The droplets can also be encapsulated in albumin or lipid shells. Droplets of nanometer size (100-200 nanometres) may also be accumulated in cancerous tissues through the EPR (enhanced permeability and retention) effect and utilized in targeted drug delivery. The droplets with and without being loaded with pharmaceutical agents may be used as a therapeutic drug delivery system in combination with ultrasound.

Cavitation is defined to include oscillation, collapse, or both of the microbubbles. Oscillation and/or collapse of the microbubbles may enhance drug delivery.

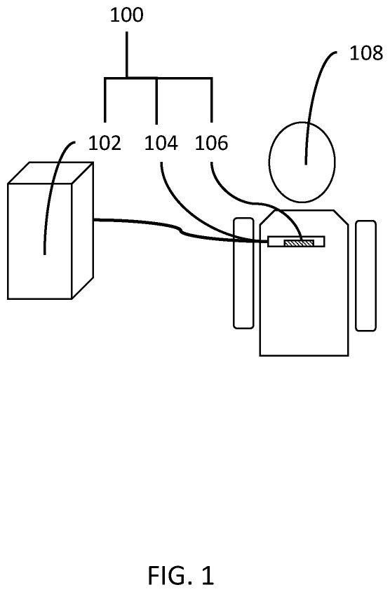

Referring now to FIG. 1, an embodiment apparatus is depicted. The apparatus 100 includes an ultrasound signal generator 102 connected to an ultrasound transducer assembly 104. The ultrasound transducer assembly 104 has at least one ultrasound transducer 106 configured to transmit ultrasound signals 202 to different parts of a lung 200.

In this example embodiment the ultrasound transducers 106 are configured to be stationary within the ultrasound transducer assembly 104. In this example, the ultrasound transducer assembly 104 is a linear-array ultrasound transducer assembly 104, which is commonly available.

The ultrasound signal generator 102 should be configured to generate ultrasonic signals 202 sufficient for ultrasonically stimulating microbubbles to enhance delivery of a pharmaceutical product to the injured lung in a human body. For example, it was found that a 1-3 MHz ultrasonic signal at 0.1 to 3 MPa may be used to induce cavitation in fluid found in an injured lung.

The ultrasonic signal generator 102 may additionally be configured to generate ultrasonic signals 202 sufficient for mapping a chest cavity. For example, a 1-5 MHz ultrasonic signal may be used to scan most larger structures (such as a heart, lung, etc) in a human chest. The ultrasound signal generator 102 may provide an ultrasound signal in the range of: Peak Negative Pressure: 100 kPa to 5 MPa; Frequency: 100 kHz to 15 MHz; and Pulse duration: 1 to 500 cycles. A skilled person would appreciate that any ultrasonic signal of appropriate power and frequency for scanning a chest cavity, inducing cavitation of microbubbles, or both could be used without departing from the scope of this disclosure.

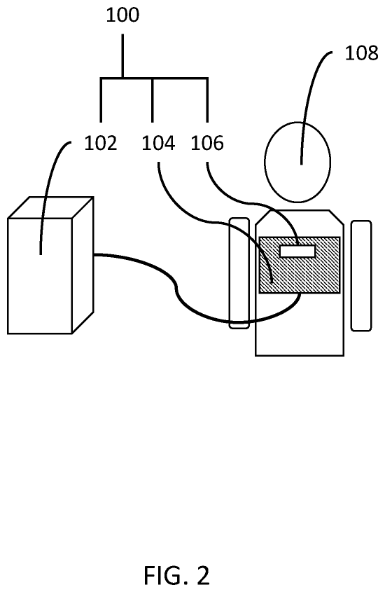

Referring now to FIG. 2, the ultrasound transducer assembly 104 is configured to house at least one ultrasound transducer 106. In this example embodiment, the one or more ultrasound transducers 106 are configured to be movable within the ultrasound transducer assembly 104. For example, in an embodiment the ultrasound transducer assembly 104 is configured to be placed on the chest of a patient such that the ultrasound transducer assembly 104 covers, at least in part, and area of the chest corresponding with at least part of the lung. The one or more ultrasound transducers 106, which are connected to the ultrasound signal generator 102, can then be moved within the ultrasound transducer assembly 104 to direct ultrasonic signals 202 to specific areas of the chest and/or lung. In another embodiment, the ultrasound transducer assembly 104 has a movement mechanism for moving the ultrasound transducer assembly 106 within the ultrasound transducer assembly. The movement mechanism may be a motor or other known mechanism for moving a ultrasound transducer 106 within a ultrasound transducer assembly 104.

In the example provided above, the ultrasound transducers 106 are configured to move within the ultrasound transducer assembly 104. For instance, the ultrasound transducers 106 may be connected to a scan line assembly (not shown) in the ultrasound transducer assembly 104. The scan line assembly (not shown) may be situated on a guide or guide rail (not shown) so that the scan line assembly (not shown) can be moved along the guide rail (not shown), thereby moving the ultrasound transducers 106 along the ultrasound transducer assembly 104. The scan line assembly (not shown) can be moved manually, automatically, or by some combination of the two. For instance, in a manual system the operator would manually move the scan line assembly to a desired position in the ultrasound transducer assembly 104 using a lever to guide the scan line assembly (not shown) along the guide or guide rail (not shown). In another example, the scan line assembly may move automatically once a signal is received from a control unit. The control unit may be configured in the ultrasound signal generator 102 or a general computing unit, for example. In this example, the scan line assembly may be configured to move along the guide rails using an electric motor.

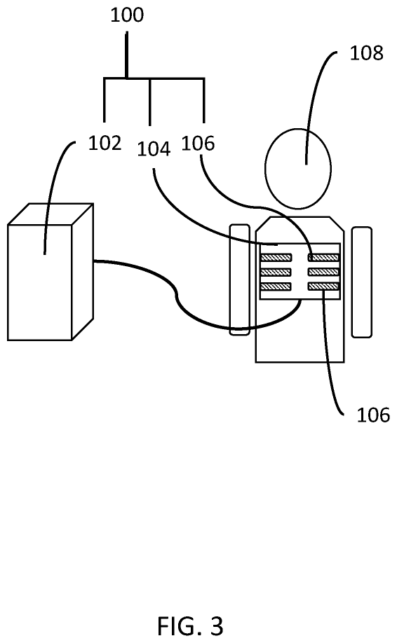

Referring now to FIG. 3, in another example embodiment the one or more ultrasound transducers 106 may be configured in fixed positions in the ultrasound transducer assembly 104. In this example, the ultrasound transducers 106 are configured so that once the ultrasound transducer assembly 104 is placed on a patient's chest, the ultrasound transducers 106 are situated at or near the space between the patient's ribs. Avoiding the ribs (and bone in general) allows the ultrasound transducers 106 to better transmit the ultrasonic waves 200 into the patient's chest cavity.

In another example embodiment, the ultrasound transducers 106 in the ultrasound transducer assembly 104 may be movable or adjustable so as to allow the ultrasound transducers 106 to be placed between the ribs of patients of different shape, size, and gender.

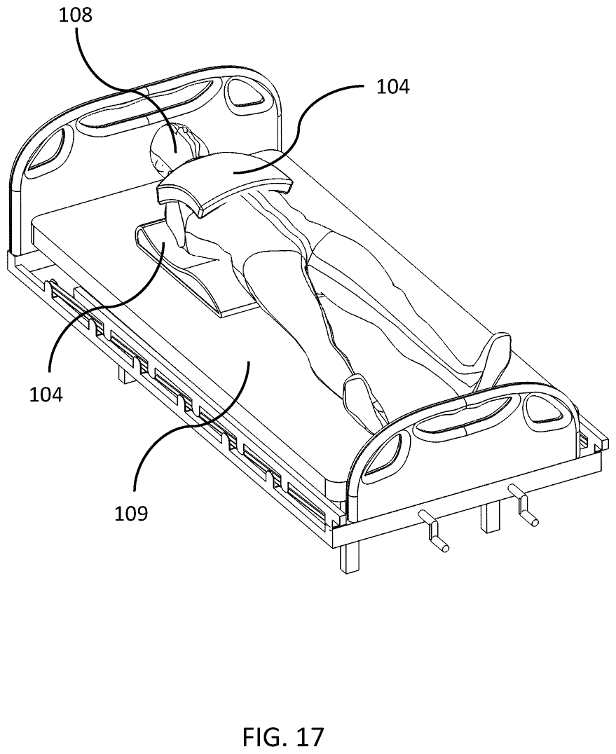

Referring now to FIG. 17, there is shown a patient 108 lying supine on a hospital bed 109 with ultrasound transducer assemblies 104 both below the chest and above the chest. In this embodiment, the ultrasound transducer assemblies 104 both below the chest and above the chest, either individually or in cooperation, are for imaging and/or treating an entire lung or both lungs. In another embodiment, a single ultrasound transducer assembly either above the chest or below the chest is configured for imaging or treating an entire lung or both lungs. In an embodiment, the ultrasound transducer assembly 104 has a flexible planar body for covering the at least part of the chest, half of the chest, or the entire chest, and without movement of the ultrasound transducer assembly, the ultrasound transducer transmits the ultrasonic signal to the pulmonary tissue. It is understood that the size of the chest for a patient may vary from that of a small child to a large man or woman.

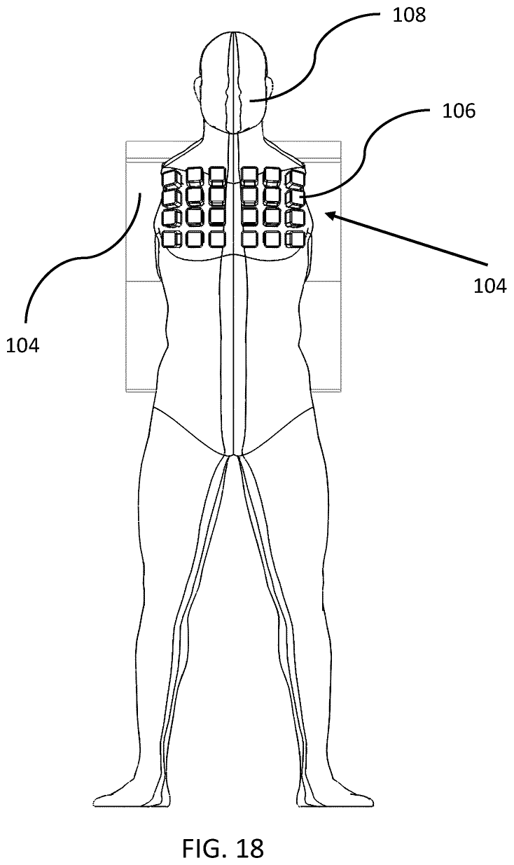

Referring now to FIG. 18, there is shown a 2D array of ultrasound transducers 106 on the top of, and the ultrasound transducer assembly 104 below, the chest of the patient 108. In this embodiment, the ultrasound transducer assembly 104 or the ultrasound transducers 106 are configured to maintain an adjustable spacing between ultrasound transducers 106 for positioning the ultrasound transducers 106 on or below the chest 500. The position may be the intercostal muscles between the ribs. The intercostal muscles transmit ultrasound signals 202 better than the ribs 201. A skilled person would understand that muscles transmit ultrasound signals better than bone. In an embodiment, the ultrasound transducer 106 has a width and a length about the size of the intercostal muscles for transmitting the ultrasonic signal between the ribs.

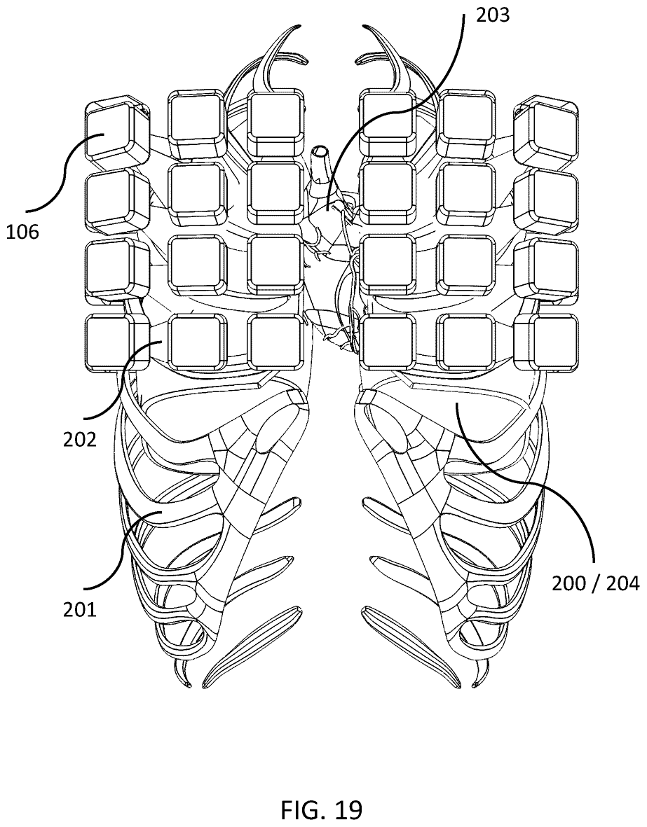

Referring now to FIG. 19, there is shown ultrasound transducers 106 emitting ultrasound signals 202 between ribs 201 towards the lung 200 and injured lung tissue 204. The ultrasound transducers 106 are programmed to not emit ultrasound signals toward the heart 203 or other organs. In an embodiment, an array of ultrasound transducers 106, the array of ultrasound transducer spaced apart a distance and each ultrasound transducer 106 is operatively connected to the ultrasound signal generator 102. In another embodiment, a 2-dimensional array of ultrasound transducers, the array of ultrasound transducer spaced apart a first distance in a first dimension and a second distance in a second dimension, and each ultrasound transducer is operatively connected to the ultrasound signal generator. In another embodiment, the ultrasound transducer assembly is configured to maintain an adjustable spacing between the ultrasound transducers. The adjustable spacing may be due to the material properties of the ultrasound transducer assembly 104, an adjustable mechanical mechanism within the ultrasound transducer assembly 104, and/or an adjustable mechanical mechanism between the ultrasound transducers 106.

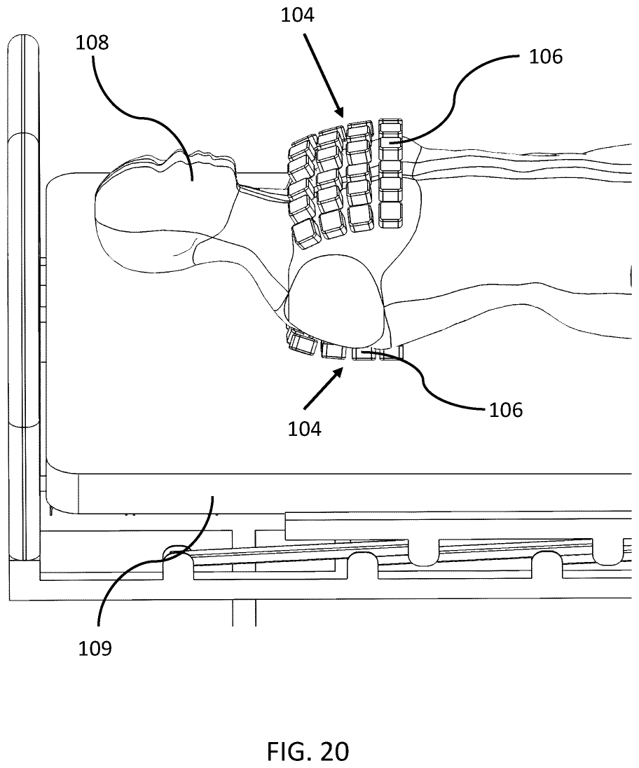

FIG. 20 shows ultrasound transducers 106 on top of and below the chest of the patient 108. In this embodiment, the ultrasound transducers are individually within an ultrasound assembly 104. The ultrasound transducers 106 and/or ultrasound transducer assemblies 104 are operatively connected to each other. In an embodiment, the ultrasound transducer or the array of ultrasound transducers is capable of imaging, treating, or both imaging and treating an entire lung or both lungs entirely.

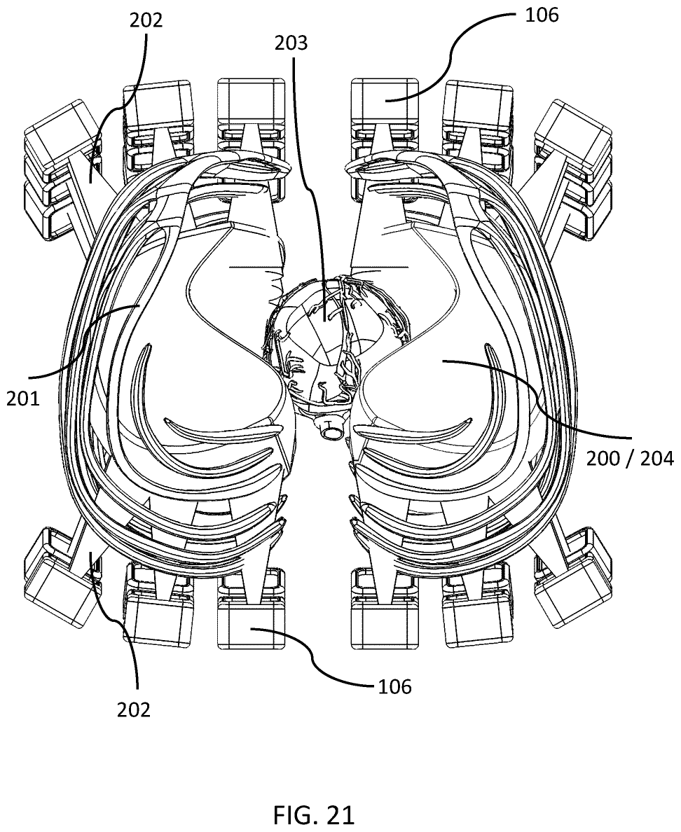

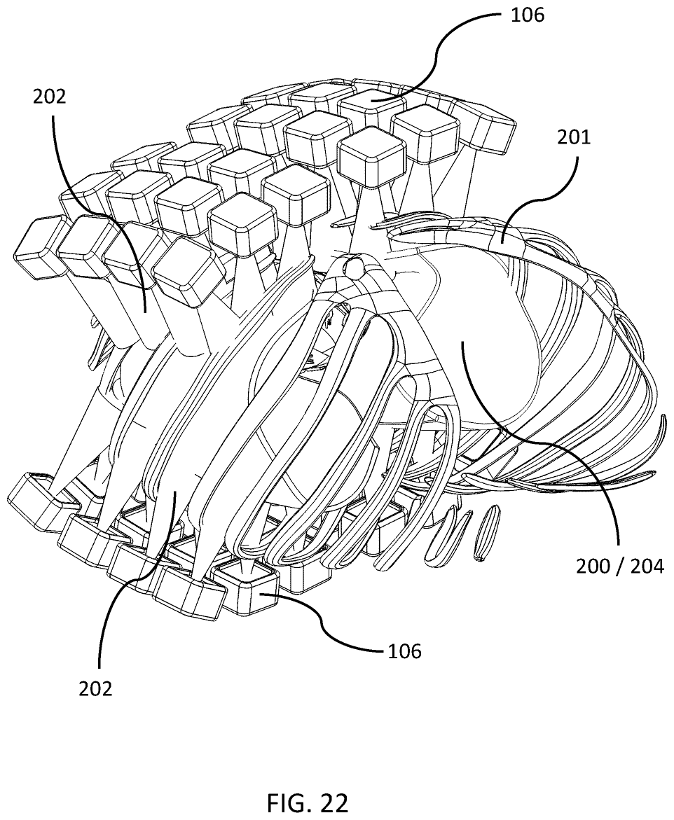

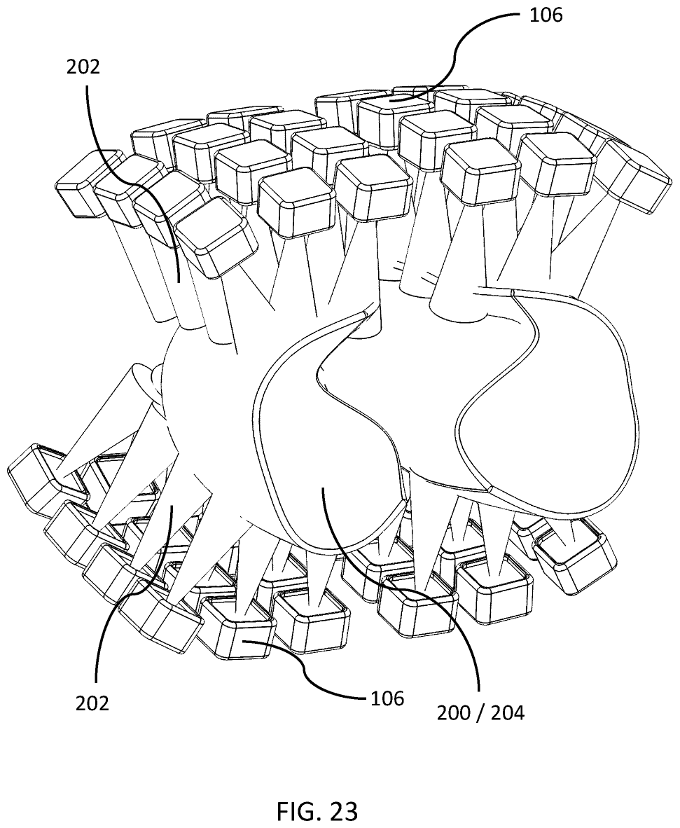

FIGS. 21, 22, and 23 show ultrasound transducers 106 transmitting ultrasound signals 202 through the ribs 201 (not shown in FIG. 23) towards the lung 200 and injured lung 204. The ultrasound transducers 106 are configured to operate individually and/or together as programmatically determined by the pulmonary ultrasound apparatus 100 (not shown) and/or the processing unit/computer (not shown). In FIG. 23, the ribs are not shown to better show the ultrasound signals 202.

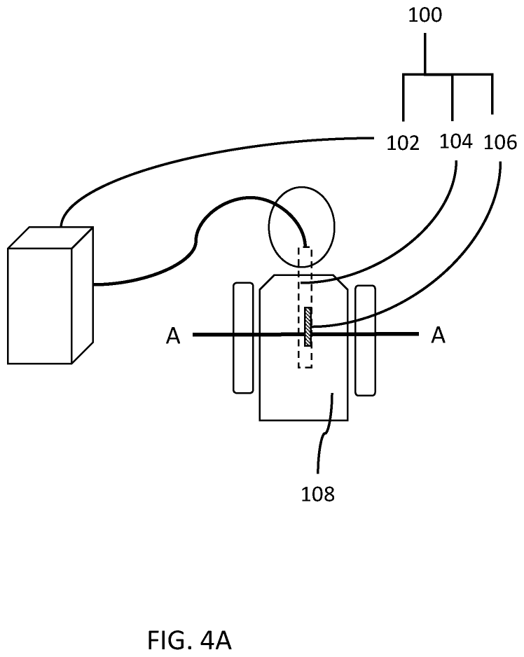

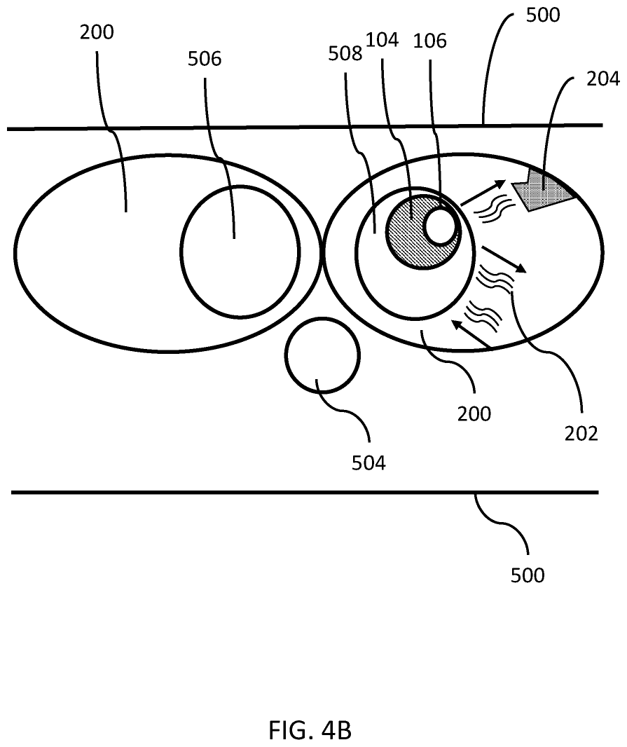

Referring now to FIG. 4A and FIG. 4B, in this example the ultrasound transducer assembly 104 is configured to be inserted endobronchially. In this example, the ultrasound transducer assembly 104 is configured to be inserted through the patient's mouth, down the patient's trachea (not shown), and into either the right bronchi 506 or the left bronchi 508 of the lung 200. In another embodiment, the ultrasound transducer assembly 104 may be inserted into the bronchi surgically in an invasive procedure. In another embodiment, the ultrasound transducer assembly 104 has an elongated body for endobronchial insertion, and the ultrasound transducer 106 is at a distal end of the elongated body for transmitting the ultrasonic signal 202 to the pulmonary tissue.

As is shown in FIG. 4B, in this example the ultrasound transducer assembly 104 is configured to contact, at least in part, a wall of bronchi so that an ultrasonic signal 202 can be transmitted and received to different areas of the lung 200.

In this example, the one or more ultrasound transducers 106 may be configured to move within in the ultrasound transducer assembly 104 so that ultrasonic signals 202 can be transmitted and received from different parts of the lung 200. In the example shown in FIG. 4B, the ultrasonic transducer assembly 104 is transmitting and receiving ultrasonic signals 202 from the lung 200, and specifically to an area having injured lung tissue 204

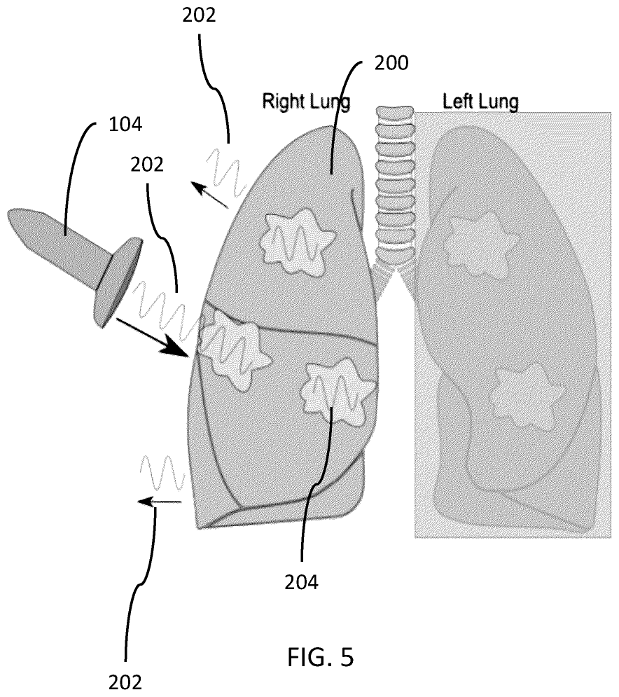

Referring now to FIG. 5, a high-level description of the system is depicted. In this example, the ultrasonic transducer (not shown), which is enclosed in an ultrasound transducer assembly 104, transmits an ultrasonic signal 202 generated by the ultrasound signal generator (not shown) towards the lung 200. Healthy parts of the lung 200 will reflect, at least in part, the ultrasonic signal 202 due to the lung (tissue) air interface. In contrast, injured lung tissue 204 will allow transmission or penetration, at least in part, of the transmitted ultrasonic signal 202. The ultrasonic transducer (not shown), as is well known in the art, is further configured to receive the reflected ultrasonic signals 202.

The system may be configured to map the lung 200 and its surrounding area using known techniques. In the example provided in FIG. 5, the ultrasound signal generator (not shown) is configured to generate an ultrasonic signal 202 appropriate for scanning the chest or thorax area.

The ultrasound signal generator may also be configured to generate an ultrasonic signal 202 appropriate for inducing cavitation of microbubbles. In the example provided in FIG. 5, the ultrasonic signals 202 penetrate, at least in part, the injured lung tissue 204. The ultrasonic signals 202 are configured to induce cavitation of microbubbles found within the injured lung tissue 204.

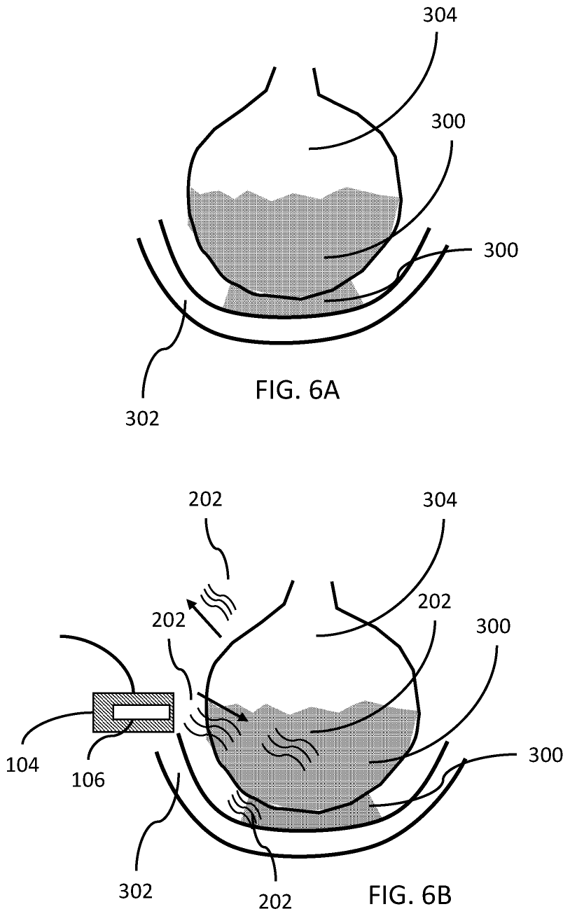

Referring now to FIG. 6A—FIG. 6D, a method for using the apparatus is provided. Referring to FIG. 6A, injured lung tissue 204 is depicted. An alveolus 304 of the lung contains fluid 300. This fluid 300 (represented by the shaded areas) may come from a variety of sources which include, but are not limited to, cell damage, leakage from the alveolar capillary 302, or secretions from cells. The areas of the alveolus 304 that do not contain fluid 300 are typically filled with air (or a gas).

In FIG. 6A, the space between the bronchial capillary 302 and the alveolus 304 also contains fluid 300. In some instances, the fluid in the alveolus 304 and the fluid in the space between the alveolar capillary 302 are the same. In other instances, the fluid 300 in the alveolus 304 and the fluid 300 in the space between the alveolar capillary 302 may be different (e.g., pus, blood, mucous, etc.).

In a healthy lung 200 gas exchange occurs, through the space between the alveolus 300 and the alveolar capillary 302. In injured lung tissue 204, however, the fluid 300 interferes in the gas exchange.

Referring now to FIG. 6B, the ultrasonic transducer 106, which is housed in an ultrasonic transducer assembly 104, transmits an ultrasonic signal 202 (generated by the ultrasonic signal generator 102) towards the lung 200. Once the ultrasonic signal 202 reaches the alveolus 304, the ultrasonic signals 202 penetrate the alveolus 304 or are reflected.

Generally, ultrasonic signals 202 penetrate and/or are transmitted, at least in part, through fluids 300. Ultrasonic signal 202 are reflected, at least in part, when they encounter a gas. In this example, the ultrasonic signals 202 are reflected by the parts of the alveolus 304 that contain a gas. The ultrasonic signals are transmitted by and/or penetrate the parts of the alveolus 304 and any other areas that contain a fluid 300 (as represented by the shaded areas).

In the step depicted in FIG. 6B, an image of the lung 200 and its surrounding area may be obtained by analyzing the reflected, absorbed, and transmitted ultrasonic signals 202 using known techniques. In the example depicted in FIG. 6B, this depiction may correspond with a scanning step whereby an ultrasonic scan of the lung 200 and its surrounding area can be taken to determine a treatment plan.

Referring now to FIG. 6C, the ultrasonic transducer 106 (which is in an ultrasonic transducer assembly 104) transmits an ultrasonic signal 202 (generated by the ultrasonic signal generator 102) towards the lung 200. In this example, the ultrasonic signal 202 is configured to induce cavitation of microbubbles once the ultrasonic signal 202 penetrates or are transmitted through the fluid 300. This cavitation is configured to induce microbubbles 306 to deliver a pharmaceutical product to the injured lung tissue 204.

It will be understood that the ultrasound signal 202 can be targeted to specific areas of the lung 200 so that ultrasonic signals 202 only induce cavitation of microbubbles at those specific areas. These methods are known, and examples of targeting techniques include, but are not limited to, ultrasonic beamforming techniques.

Referring now to FIG. 6D, the ultrasonic transducer 106 (which is in an ultrasonic transducer assembly 104) again transmits an ultrasonic signal 202 (generated by the ultrasonic signal generator 102) towards the lung 200. This step may be used to determine the effects of the cavitation step (i.e., FIG. 6C) on, and the area surrounding, the injured lung tissue 204.

In this step, an operator may review the results of the scan from FIG. 6A and the results of the scan from FIG. 6D to determine any changes in echogenicity that resulted from the cavitation step (i.e., FIG. 6C). In another example embodiment, the system may be configured to automatically compare the results of the scan from FIG. 6A and the results of the scan from FIG. 6D. For instance, a computing unit may be configured to determine, algorithmically and automatically, the echogenicity changes made in the cavitation step (i.e., FIG. 6C). A skilled person would understand that alternative methods of comparing the results of ultrasound scans can be used without departing from the scope of this disclosure.

In another embodiment, a pre-scan of tissue or injured lung tissue 204 by ultrasound may identify echo regions showing fluid filled regions, and then a post-scan may identify less echo regions showing less fluid filled regions. For example, the change in the amount of fluid, as shown by the change in echogenicity, in the fluid filled regions may be due to the injured lung tissue responding to the pharmaceutically active compound delivered by cavitation of the microbubbles (i.e. less fluid in healthier or less injured lungs). In another embodiment, the microbubbles may be detected in the tissue or injured lung tissue 204 by the echogenicity of the tissue without microbubbles as compared to the echogenicity of the tissue with microbubbles.

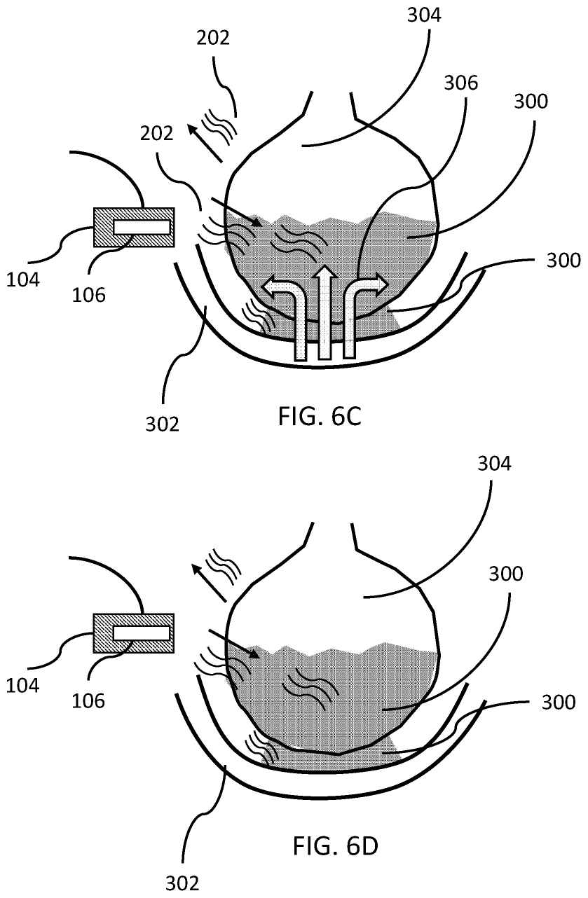

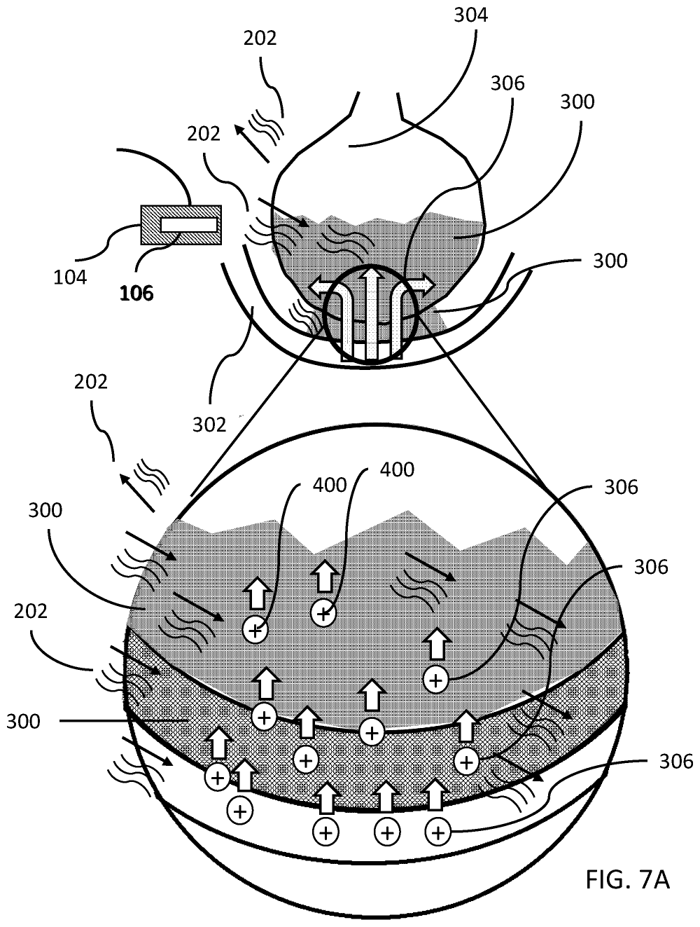

Referring now to FIG. 7A, a close-up view of FIG. 6C is depicted. In this example, a pharmaceutical product 400 is contained within microbubbles 306. The pharmaceutical product 400 is released once the microbubble 306 disintegrates, implodes, cavitates, or otherwise collapses. The microbubbles 306 may be introduced from an external source. For instance, microbubbles 306 can be introduced intravenously so that they travel throughout the bloodstream, including into the alveolar capillaries 302. Microbubbles 306 may also be injected directly into the part of the lung 200 containing injured lung tissue 204. A skilled person would understand that other ways of introducing microbubbles 306 to the lung, the alveoli, or areas near the lung and/or alveoli, can be contemplated without departing from the scope of this disclosure.

Once the ultrasonic signal 202 is applied in the area containing the injured lung tissue 204 and the microbubbles 306, the ultrasonic signal 202 induces cavitation of the microbubbles 306, this may lead to the enhanced uptake of pharmaceutical product in the injured lung tissue 204. A skilled person would understand that alternative ways of using ultrasound to cavitate microbubbles may be used without departing from the scope of this disclosure.

Referring now to FIG. 7B, an alternate close-up view of FIG. 6C is depicted. In this example, a pharmaceutical product 400 is proximate to the microbubbles 306. Similar to FIG. 6A, the microbubbles can be delivered in a variety of methods including, but not limited to, intravenously.

Once the ultrasonic signal 202 is applied in the area containing the injured lung tissue 204 and the microbubbles 306, the ultrasonic signal 202 induces cavitation of the microbubbles 306 leading to enhanced uptake of the pharmaceutical product in the injured lung tissue 204.

Any pharmaceutically active molecule 400 that can be deployed within or proximate to a microbubble 306 can be used without departing from the scope of this disclosure. Examples of a pharmaceutical product 400 include, but are not limited to, small molecules such as antibiotics and chemotherapeutics, nucleic acids such as micro-RNA, DNA and modified nucleic acids, proteins, enzymes, steroid, antivirals, and other small molecule drugs, including CRISPR components. Some specific examples of antibiotics that may be co-formulated with the microbubble are gentamycin, tobramycin, cefazolin, piperacillin, and tazobactam, vancomycin, and carbapenems. A composition comprising two or more types of microbubbles can also be used, wherein each microbubble has a different composition, potentially formulated with and without, or with different pharmaceutically active species. Microbubbles may also be formulated with two or more different pharmaceutically active species, either incorporated into or mixed with the microbubble. Methods of deploying a pharmaceutical product proximate to a microbubble include, but are not limited to, including the pharmaceutical product in the solution containing the microbubbles, introducing the pharmaceutical product (e.g., via site injection, intravenous injection, etc.) separately to the injured area and/or the area containing the microbubbles, or a combination of the two.

In addition to small molecules, miRNA can be delivered in combination with microbubbles to an edemic lung to regulate endothelial function, enhancing endothelial repair and improve barrier function. miRNAs are short (20-22 nucleotides), non-coding RNA sequences that regulate post-transcriptional gene expression. miRNAs are attractive therapeutic tools because each individual miRNA sequence regulates a large number of genes, reducing the problem of target redundancy that occurs when a single gene is manipulated. miRNAs typically downregulate target mRNAs by impairing protein translation or inducing mRNA degradation. Less commonly, miRNAs can upregulate downstream mRNA sequences. miRNA can also target nuclear transcription factors, amplifying their downstream effects. In addition to regulating multiple proteins, one advantage of miRNA as therapeutic agents is that they are relatively stable chemically and are highly conserved among species. Furthermore, synthetic RNA molecules have now been created that either antagonize specific miRNA sequences or mimic them (antagomirRs and miRNA mimetics, respectively). It is also possible to inhibit miRNA-binding to a specific target mRNA sequence (e.g. Blockmirs) All of these permutations add to the potential therapeutic utility of miRNAs in pulmonary edema. In an embodiment, antagomirs and blockmirs to miR27a and miR146b are the pharmaceutically active molecule.

Specific miRNAs have been reported to regulate critical aspects of endothelial barrier function, including endothelial activation, junctional protein modification, and endothelial apoptosis. For example, miRNA-181b is rapidly down-regulated by endothelial cells that have been exposed to the inflammatory cytokine TNFa. Overexpression of miR-181b using a miR-181b mimic suppressed endothelial NFκB activation and decreased endotoxin-induced lung injury. The effect of this miRNA on endothelial NFκB is intriguing given that excessive NFκB activation in the endothelium is known to induce vascular leakage. Furthermore, circulating miR-181b levels are reduced in patients with sepsis/ARDS compared to ICU controls, suggesting that supplementation of this miRNA in ARDS may be beneficial. Indeed, it has been shown that miR-181b induces expression of the junctional protein VE-cadherin during embryonic development.

Another potentially important sequence is miRNA126-3p. This miRNA is highly enriched in endothelial cells and its loss causes vascular leak through impaired vascular endothelial growth factor (VEGF) signaling Importantly, miRNA126 has been shown to be downregulated in animal models of lung injury. Although enhanced VEGF signaling induces angiogenesis (and characteristically leaky blood vessels), it has been shown that miRNA126 over-expression also enhances activity of the Tie2 receptor in response to angiopoietin-1. The coordinated expression of VEGF and Ang1 leads to blood vessel maturation. As outlined earlier, the Tie2 signaling axis is known to be impaired in severe infections and inflammation, making it an important therapeutic target. Thus, supplementation of miRNA126-3p to injured regions of lung may improve recovery.

There are also several miRNA sequences that have been described to regulate expression of VE-cadherin, the major constituent of endothelial adherens junctions and a primary determinant of barrier function. For example, inhibition of miR-27a using a Blockmir against the binding site in VE-cadherin mRNA specifically prevented downregulation of VE-cadherin and attenuated vascular permeability in a model of limb ischemia. It has further been observed that over-expression of miR-27a can cause a reduction in VE-cadherin protein levels and knockdown had the opposite effect. Thus, it is possible that inhibition of miR27a in the injured lung (using antagomirRs) can contribute to restoring lung endothelial barrier function.

Another candidate for targeted delivery to the injured lung is miRNA150-5p. This miRNA is decreased in patients with sepsis and its deletion caused a persistent increase in angiopoietin-2 levels (Ang2). Ang2 is an endogenous competitor of Ang 1 that binds to the Tie2 receptor and destabilizes the endothelial barrier through disruption of adherens junctions. Deficiency of miR-150-5p is associated with impaired re-annealing of VE-cadherin and impaired recovery from endotoxin-induced endothelial leakage in vitro. Conversely, restoring miRNA150-5p expression in miR-150 null (knockout) mice has been shown to reduce lung edema from endotoxin. Most convincingly, supplementation of a mimetic of miRNA150-5p led to decreased Ang2 levels and lower mortality from endotoxemia. Thus, delivery of miRNA150-5p to the injured lung may also be beneficial in ARDS. Testing whether targeted delivery of miRNA150-5p or antagonists of miR27a improve the outcome of ARDS

Ultrasound has the capability of reaching most of the lung. In particular, thoracic ultrasound has a maximum tissue penetration depth of 10 cm. This suggests that much of the lung could be targeted for drug delivery by the present method. For instance, if the chest circumference is 38 inches, the chest radius would approximate 6 inches or 15 cm. Furthermore, endobronchial ultrasound, which is now used routinely, would also permit access to the innermost regions of the chest. Portable ultrasound units are now found in almost every intensive care unit (ICU) in the country and all new physicians now receive training in this technique. Compared to other imaging modalities, ultrasound also requires very little movement of the patient. Repeated ultrasound examination has been found to safe and relatively inexpensive, providing for feasible repeated administration. A variety of different treatment algorithms can be used, including varying time, pulse strength, location, duration of treatment, etc. In particular, a number of different variables can be changed to optimize patient treatment and how apparatus can be tuned to provide treatment variability in these variables.

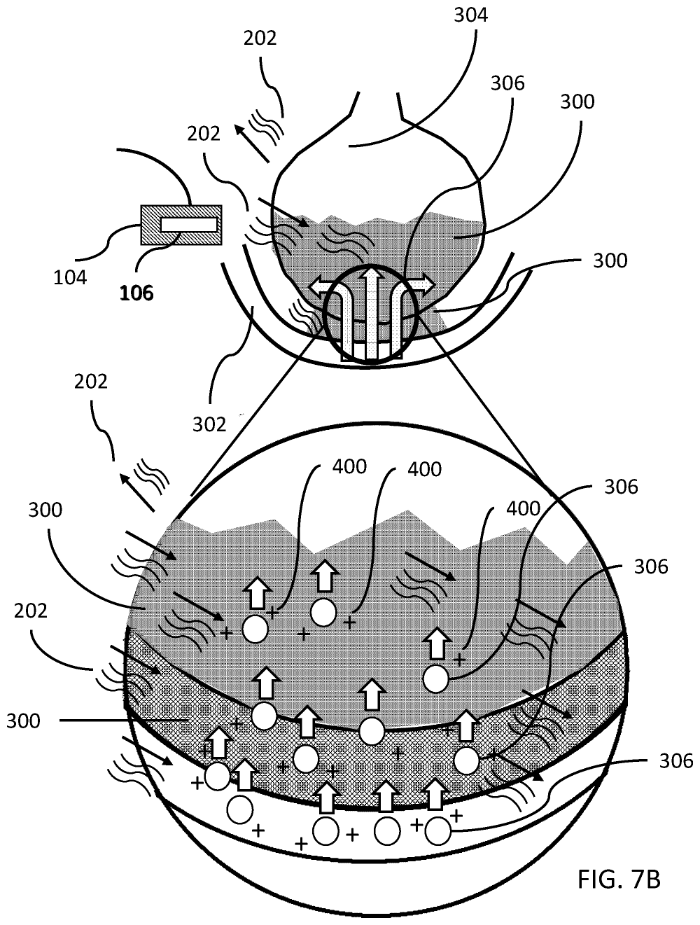

Referring now to FIG. 8, a flow diagram depicting an example method of using the apparatus is provided. In the example depicted in step 600, once the microbubbles 306 have been delivered to the area proximate the injured lung tissue 204, an ultrasonic signal 202 is applied to the injured lung tissue 204 and its surrounding area. The ultrasonic signal 202 induces cavitation of the microbubbles 306, leading to the enhanced uptake of pharmaceutical product into the injured lung tissue 204. The pharmaceutical product 400, which can either be proximate to the microbubbles 306 or within the microbubble 306 itself, is delivered into the injured lung tissue 204.

In the example depicted in step 602, once the pharmaceutical product 400 has been delivered to the injured lung tissue 204 a second ultrasound signal 202 is sent, among other places, to the area surrounding the injured lung tissue 204 that was previously treated. This ultrasound signal 202 is configured to scan the area in and around the previously treated injured lung tissue 204 in order, for example, to show an operator the effect of the delivered pharmaceutical product 400 on the injured lung tissue 204.

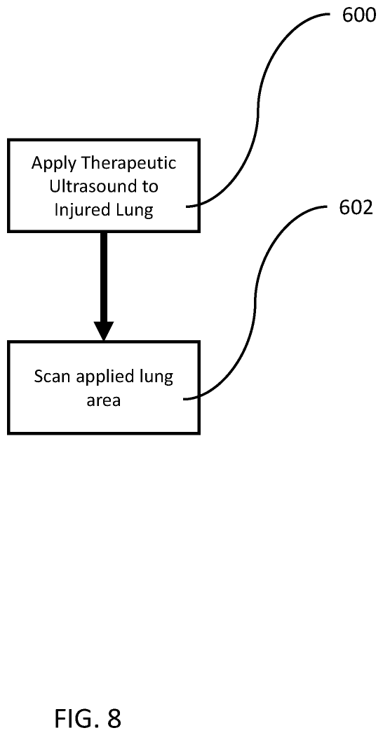

Referring now to FIG. 9, a flow diagram depicting an alternate example embodiment of using the apparatus is provided. In the example depicted in step 700, the body cavity of the patient is scanned, at least in part, to locate and identify injured lung tissue 204. If, in step 702, no injured lung tissue 204 is detected, the ultrasound transducer 106 is moved, as shown in step 704, within the ultrasound transducer assembly 104 so that a different part of the body cavity can be scanned. The ultrasound transducer 106 can be moved either manually, automatically, or a combination of the two. In the embodiment where the ultrasound transducer assembly 104 includes multiple ultrasound transducers 106, an alternate ultrasound transducer 106 corresponding to a different area of the chest cavity can be selected.

Among other things, step 702 and step 704 helps to avoid scanning and/or transmitting ultrasonic signals 202 to parts of the body that do not contain injured lung tissue 204. These body parts can include, but are not limited to, healthy lung tissue, the heart, and other internal organs.

The scanning step 700 is repeated until injured lung tissue 204 is detected 702. Once injured lung tissue 204 is detected, the therapeutic step 600 and scan step 602 similar to the ones depicted in FIG. 8 are performed. In the second scanning step 602 in this embodiment, if injured lung tissue 204 is still detected 702 the therapeutic 600 and scan 602 steps may be repeated. If, however, the second scanning step 602 does not detect injured lung tissue 204, then the ultrasound transducer 106 may be moved, an alternate ultrasound transducer 106 selected, or scanning stopped (not shown), as the case may be.

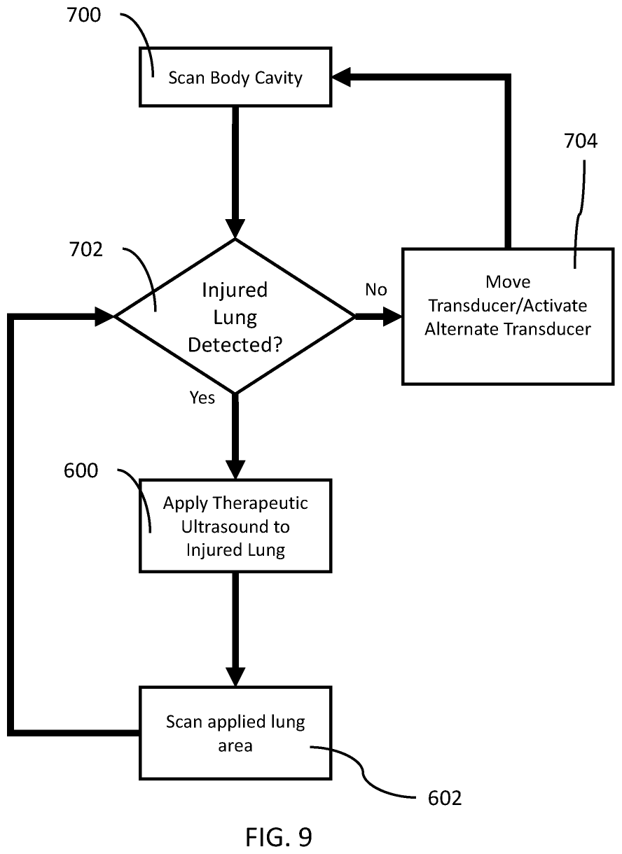

Referring now to FIG. 10, a flow diagram depicting an alternate example embodiment of using the apparatus is provided. In this example, the additional step of detecting microbubbles 800 is provided between the therapeutic step 600 and injured lung tissue detection step 702. If microbubbles are detected 800 then the therapeutic step 600 is performed. If, however, microbubbles are not detected 800 then the ultrasound transducer 106 is moved, an alternate ultrasound transducer 106 selected 704, or a delay happens (not shown), as the case may be. The additional step of detecting microbubbles 800 prevents the therapeutic step 600 from being performed if there will be no effect.

A skilled person understands that detection of microbubble cavitation is known. Also, there are several parameters associated with integrated cavitation dose (energy associated with the cavitation activity of the microbubbles) either over the whole transducer bandwidth or within a certain frequency bandwidth (e.g. harmonic, ultra-harmonic, and sub-harmonic bands). Some of these parameters may be an optimal indicator of biological effects in the lungs associated with microbubble cavitation.

It will be understood that the scanning, therapy, and decision steps provided above may be performed by an operator. It will also be understood that the scanning, therapy, and decision steps may be automated. A skilled person would understand that a processing unit (or computer) could be used to automate, at least in part, the steps described herein without departing from the scope of this disclosure. For instance, a computer/processing unit could be used to compare the results of the body cavity scan 700 and the post-treatment scan 602 to determine the results of the treatment step 600 without the need for operator intervention. Similarly, a computer/processing unit may be used to detect injured lung tissue 702 and/or microbubbles 800 once the first scan 700 has been performed. A computer/processing unit may also be used to determine a treatment plan for applying therapeutic ultrasound to the injured lung tissue 600. A computer/processing unit may also be used to move or activate the ultrasound transducers 704. In an embodiment, the treatment method controls the positioning and/or movement of the transducer or transducers, and transmitting of the ultrasound signal through the transducer or transducers, including the ultrasound parameters, and the method is implemented through software executing on a computer.

Specific parameters to be considered are: size of the patient; size and/or location of ribs; size and/or location of organs (e.g. heart and other organs); anatomical and physiological parameters such as size, weight, and shape of the patient, chest, and chest cavity; number of transducers; position of transducers; and activation sequence and/or pattern of transducers.

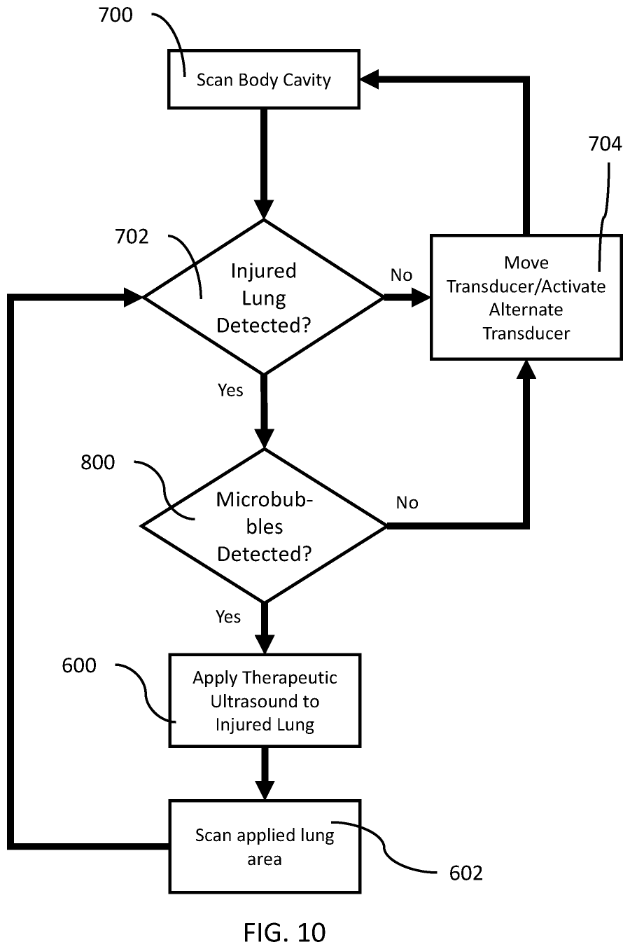

FIG. 11 illustrates an ultrasound mediated pulmonary drug delivery approach using microbubbles. A hand-held ultrasound probe is applied to the surface of the chest. In a normal lung (top), air-filled regions of the lung cause scattering or blocking of the ultrasound waves (arrows). In contrast, damaged areas of the lung with ARDS (bottom) are filled with fluid and allow penetration of the ultrasound beam. In addition, by targeting the ultrasound probe to the chest, the ultrasound energy is focused on the thorax, preventing an effect on other organs. Note the marked heterogeneity of the lung in ARDS, with black (air-filled) areas of the lung interspersed with white (fluid or pus-filled) regions. Conventional therapeutic approaches cannot specifically target the damaged regions of the lung.

Thoracic ultrasound can be used safely, and in combination with microbubbles and a pharmaceutically active species, results in enhanced delivery of the pharmaceutically active species to consolidated/non-aerated and injured lung tissue. A variety of pharmaceutically active species can be co-formulated or concurrently delivered with microbubbles to provide conditions for improved delivery of the pharmaceutically active species to edemic lungs. A variety of potential drug molecules may be effectively delivered using ultrasound microbubble treatment. Non-limiting examples of pharmaceutically active compounds which may be effectively delivered to the lung in combination with ultrasound microbubbles include: antibiotics including aminoglycosides and vancomycin; antivirals such as Tamiflu; endothelial barrier-enhancing drugs such as vasculotide, adrenomedullin, fasudil, imatinib, atrial natriuretic peptide, and SIP; and Keratinocyte growth factor (KGF). In addition, genetic material can be co-administered or co-formulated with microbubbles to provide effective delivery of the genetic material to edemic lung tissue. Non-limiting examples of genetic material that can be delivered include miRNA.

Intravenously delivered miRNA without microbubbles has a very short half-life and will not be preferentially deposited in the lung. The concomitant use of miRNA and microbubbles, together with directed ultrasound application to lesions of pulmonary edema provide miRNA to the lung at the site of action, and in the window of physiological stability of miRNA. Thus, the ultrasound microbubble-mediated strategy can be viewed not only as a therapeutic approach for longer half-life drug molecules such as antibiotics and antivirals, but also as an efficient method for delivering genetic material to the site of lung injury, since expression of delivered genes will be highly enriched in these injured regions.

Microbubbles are comprised largely of lipids which form a gas-filled bubble and can be formulated in a variety of ways based on their use in clinic and their surface charge. One example of microbubbles are Definity®, a Health Canada-approved microbubble preparation that is used for diagnostic imaging in ICU patients. The surface of the Definity microbubbles has a slightly negative charge. Another example of a microbubble preparation is custom-made, composed of polyethyleneglycol-40 stearate, distearoyl phosphatidylcholine and distearoyyl-3-trimethylammoniumpropane with decafluorobutane gas, and has a positively-charged surface which favours binding to nucleic acids. It has been found that these microbubbles are especially well suited for co-formulation and delivery with miRNA. A range of doses (dose-response) of microbubbles can be used to optimize delivery of the pharmaceutically active material while maintaining patient safety. As coupling between the miRNA and the microbubbles, which is based on charge, the binding capacity of the microbubbles can be worked out for miR-126-3p and can be easily determined for the other sequences using the same method. In particular, charge-coupling of miR-126-3p and microbubbles significantly prolongs the half-life of the miRNA in the circulation in the absence of ultrasound stimulation (e.g. at least 3 hours after injection). In contrast, free miRNA is cleared from the circulation within minutes. This stable coupling indicates that the microbubbles cavitated by lung ultrasound are likely to have miRNA bound to them and that any free miRNA is unlikely to be important.

Emerging literature indicates the importance of loss of endothelial integrity in the pathogenesis of ARDS, and microRNAs 181b, 126-3p, 150 and 27a are known to have endothelial effects. Lung ultrasound and microbubbles may also be used to enhance the targeting of other miRNAs, some of which will target epithelial cells, leukocytes and other contributors to the pathogenesis of lung injury. In addition, combination therapy using more than miRNA sequence, or a miRNA sequence in combination with a pharmaceutically active small molecule or protein. Obtaining binding of miRNA sequence to the microbubbles at stoichiometrically-constant proportions may be addressed in part by administering combinations of microbubbles, each bound to a single miRNA.

### EXAMPLES

In order that the invention described herein may be more fully understood, the following examples are set forth. It should be understood that these examples are for illustrative purposes only and are not to be construed as limiting this invention in any manner.

### Example 1—Murine Microbubble Model

To confirm the safety of microbubbles and ultrasound-mediated cavitation in this setting, oxygen saturation was measured by pulse oximetry before, during and 2 hours after intravenous injection of microbubbles (IX 10⁹ microbubbles) and thoracic ultrasound administration. Hypoxemic mice are observed before, during and after injection of microbubbles. Controls were done with and without the administration of ultrasound to the thorax.

Balb/c mice (male, age 12-16 weeks) were infected with 0.5×10⁷ cfu E. coli intratracheally and developed acute hypoxemia and hypothermia. Arterial oxygen saturation (Sp0₂) was measured 3 hours post-infection by pulse oximetry. Mice were then anesthetized with inhaled isoflurane and received 4 litres 0₂ to immobilize them for the duration of microbubble injection and the administration of thoracic ultrasound (USMB). A clinical ultrasound machine (Sonos 5500, Phillips Healthcare) and its S3 phased array ultrasound transducer were used to induce microbubble cavitation and facilitate gene/drug delivery. For delivery, the ultrasound probe is positioned over the murine thorax and set to release single frames of high power (transmit frequency 1.3 MHz, 67V, 0.2 W, mechanical index 0.9, peak negative acoustic pressure −900-1200 kPa) at a pulsing interval of every 5 seconds. Microbubble and drug/gene complexes administered intravenously were circulating freely through the systemic circulation, during which time triggered ultrasound is simultaneously applied over the anterior thoracic cavity to enable microbubble cavitation and targeted lung delivery of the pharmaceutically active species.

Supplemental oxygen was removed immediately after ultrasound administration (5 minutes) and animals were then placed on room air. Sp0₂ was monitored during ultrasound treatment, immediately after ultrasound treatment, and two hours after ultrasound treatment, while animals were on room air. Hypoxemia is noted which occurs within 3 hours of the infection (*p<0.05 by Bonferroni post-tests vs. other groups after significant two-way ANOVA) and the stable 0₂ saturation during and after USMB. Data are mean and SEM, with n as numbers of mice. This suggests that USMB is well tolerated. Pulse rate and arterial oxygenation saturation was also measured continuously, and mice were monitored for oxygen saturation, signs of respiratory distress and overall activity during and after the experiment.

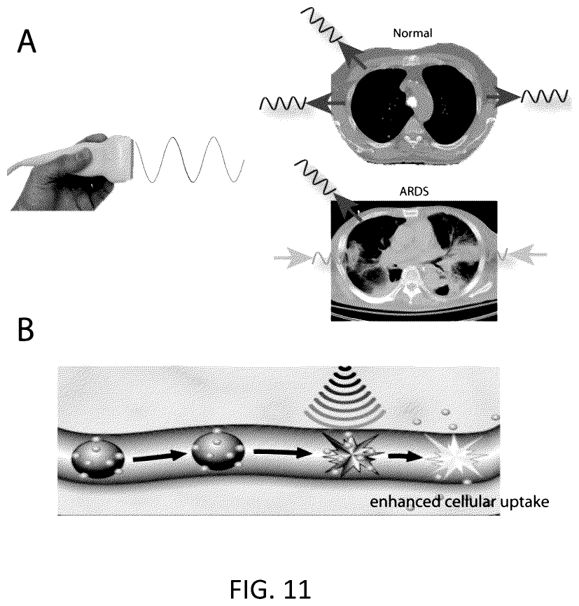

FIG. 12 is a graph illustrating pulmonary oxygen saturation in the presence and absence of microbubble ultrasound treatment. The observed rise in Sp0₂ is due to the USMB treatment. Microbubbles and ultrasound were well tolerated, with the treatment resulting in no change in arterial oxygenation.

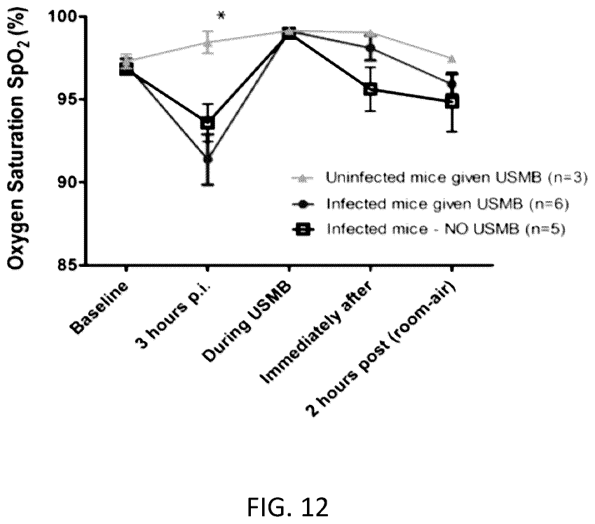

Histological examination confirmed extensive neutrophilic lung injury that was heterogeneously distributed. FIG. 13 is a set of histopathological images of lung sections of mice from this experiment, showing representative histopathological images (hematoxylin and eosin stain) from lung sections of uninfected (B) and E. coli-infected mice (C-E). Note the alveolar infiltrate and patchy distribution of the disease (e.g. panel C). Ultrasound and microbubbles (USMB) had no effect on histological lung injury (magnified view, E v. D). The histologic appearance of USMB treated lung is shown in FIG. 13 panel E. Mice that received microbubbles and thoracic ultrasound (n=6) were indistinguishable from untreated and infected mice (n=5). Histologic analysis of the lungs, hearts and brains of treated vs. control animals as well as long-term follow-up (i.e. days after exposure) of oximetry and animal behavior can also be done. If oxygenation is acutely decreased by USMB, a lower dose of microbubbles may be used to retain pulmonary oxygenation. These preliminary data are with consistent with extensive clinical experience in ICU patients and suggests that USMB is safe for thoracic use.

### Example 2—Treatment cf Murine E. coli Infection with USMB

To establish whether USMB enhances antibiotic delivery to the injured lung, gentamicin (1.5 mg/kg) was administered to mice by intraperitoneal injection 6 hours after infection with E. coli to determine whether USMB conferred any additional benefit to treatment with antibiotics or microbubbles alone. This relatively low dose of gentamicin was chosen as it is relatively slow and achieves incomplete clearance of lung pathogens and thus allows any benefit of USMB to become apparent. In humans, high doses of gentamicin are known to cause nephro- and ototoxicity, making it contraindicated for human use at high doses.

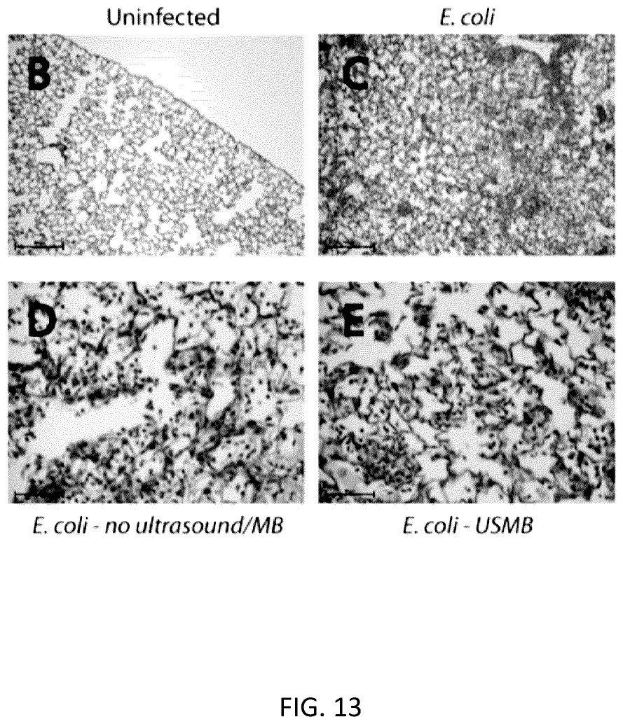

C57BL/6 mice were infected intratracheally with 1×10⁷ colony forming units (cfu) of E. coli and received 1.5 mg/kg of gentamicin by intraperitoneal injection 6 hours later. Thirty minutes later, the mice were then injected intravenously with 1×10⁹ microbubbles (Definity™) by tail vein. (Definity™ is an approved microbubble preparation that is used for diagnostic imaging in intensive care unit patients.) This was followed by thoracic ultrasound administration for five minutes. The ultrasound treatment conditions were the same as those used in Example 1. Eighteen hours later, mice were euthanized by cervical dislocation and lungs were homogenized for cfu. Lung homogenates were plated on agar to measure bacterial growth. Each experiment was repeated twice with 3 mice per group. Control mice received microbubbles and antibiotic without ultrasound (MB+antibiotics) or antibiotics alone (PBS and antibiotics). Other control mice received no antibiotics. FIG. 14A shows a graph of this experiment. A second replicate was performed a week later, the results of which are shown in FIG. 14B. Data are mean plus SEM (SD if <3 mice) with each dot representing a separate animal. Note the >1 log-fold reduction in cfu in the mice receiving USMB with antibiotics (left-most group) compared to microbubbles/antibiotics alone. An ELISA performed on lung homogenates and plasma measures gentamicin levels.

While gentamicin alone caused a reduction in bacterial colony forming units (cfu), USMB caused a 10-fold reduction in bacterial growth in both replicates. Administration of microbubbles and antibiotics without ultrasound conferred no benefit over antibiotics alone, as observed in FIGS. 14A and 14B. The used ultrasound treatment settings have been shown to be safe and result in enhanced delivery of antibiotics/miRNA to consolidated/non-aerated (hence injured) lung tissue. These data strongly support the use of USMB to enhance antibiotic delivery in pulmonary edema, and in ARDS in particular.

### Example 3—Murine Influenza a Treatment Microbubble and miRNA

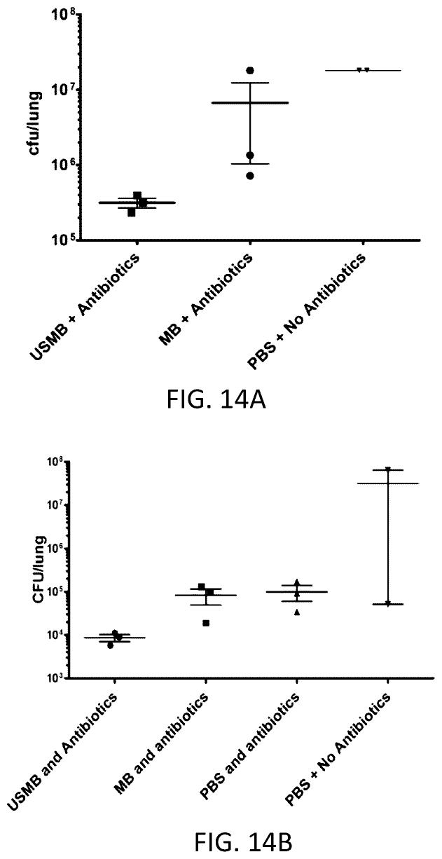

The effect of ultrasound microbubble (USMB) treatment on the deposition of circulating miRNA into the lung was tested to establish that USMB can be used to enhance gene delivery. Nine mice were randomly divided into three groups. In one group 150 μL of saline was introduced intratracheally into the lungs of C57BL/6 mice to mimic severe pulmonary edema. Mice were then injected intravenously with 1×10⁹ microbubbles tagged or charged with miRNA-Alexa Fluor 555 (A555), a fluorescent miRNA with a Cy3 (red) tag by tail vein. This was followed by thoracic ultrasound administration for 5 minutes. Control mice received the microbubbles alone without ultrasound. Mice were then euthanized and lung sections were prepared for immunofluorescence analysis imaging. Images were acquired by fluorescent microscopy of ten random sections of each lung under identical microscope settings. Red fluorescence (arbitrary units, AU) was quantified by ImageJ. The results are shown in FIG. 15 is a graph showing USMB enhanced deposition of miRNA in edematous mouse lung (note the log scale). Data are mean plus SET with each dot representing the lung from a separate animal. Note the much higher fluorescence in USMB-treated and saline-instilled lungs (triangles, with fold-increase over matching controls indicated). Although more replicates and controls are needed, significantly more red fluorescence (i.e. Alexa 555 emission) was observed in saline-instilled lungs than healthy controls.

### Example 4—Influenza a Murine Infection

Influenza A (H3N2) is the commonest subtype associated with complications and death in humans. In mice, influenza A (H3N2) is a highly lethal model and 90-100% of mice die within 8 days after infection. Infected mice develop progressive hypoxemia and lung edema in association with weight loss and hypothermia. In this model, the important contribution of the lung endothelium to determining the outcome of severe influenza has been observed. Because mortality in the mice occurs 6-8 days after infection, this model is useful to assess the effect of targeted delivery of miRNA on the outcome of ARDS, as there is enough time for miRNA-targeted gene transcripts to alter the course of the disease.

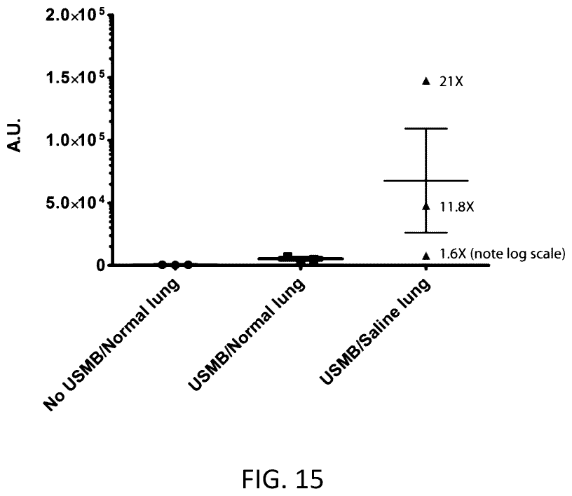

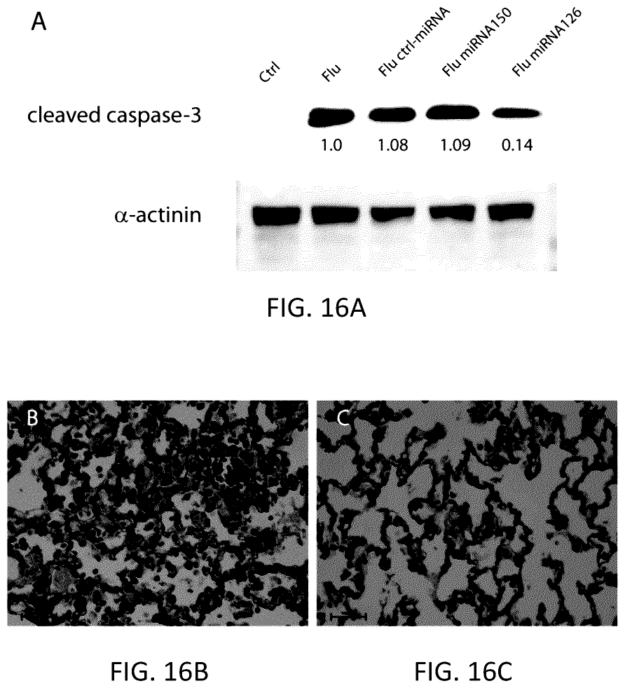

The use of miRNA 126-3p was demonstrated in vitro for injured lung for the treatment of ARDS. Primary human lung microvascular endothelial cells were transfected with miRNA or control for 48 hours followed by infection with influenza A X31 (H3N2) at a multiplicity of infection of 0.1.24 hours later, cell lysates were probed for cleaved caspase-3 as a measure of apoptosis. a-actinin is the loading control. As shown in FIG. 16A, treatment with miRNA-126-3p decreased endothelial apoptosis (quantification by photon capture). FIGS. 16B and 16C are histopathological images of lung sections (hematoxylin and eosin stain) from C57BL/6 mice infected with human influenza A. Neutrophilic infiltrate, alveolar protein and hemorrhage, as well as marked heterogeneity of injury can be observed, with tissue abnormality and damage more severe in FIG. 16B vs. FIG. 16C. This demonstrates that miRNA126-3p in combination with USMB treatment is capable of reducing influenza-induced lung endothelial apoptosis, supporting the rationale for its use in vivo. Ultrasound and microbubble administration therefore results in significantly more deposition of miRNA in the setting of pulmonary edema compared to normal, aerated lung. Other genetic material can be delivered selectively to the injured regions of the lung. In particular, some other non-limited examples of potential miRNA to deliver via USMB are 126-3p, 181b, 150, and antagomiR to miRNA27a. Other potential downstream gene targets are also envisaged, such as, for example, VE-cadherin, Tie2, Rac GTPase, RhoGTPase, PIK3R2, Caspase3, Caspasel, HMGB1, CD36, JNK1, and VE-PTP.

The effect of the individual miRNA sequences in vitro can be established using primary human lung microvascular endothelial cells. miRNA transfected into cells and successful transfection can be verified by pPCR of the miRNA and its downstream targets (e.g. downregulation of PIK3R2 by miRNA 126-3p). The effect of the miRNA on endothelial barrier function to ions and macromolecules can further be quantified by transendothelial electrical resistance (TEER) measurements and transwell assays using dextran tracers, respectively. The effect of miRNA on influenza-induced endothelial apoptosis may also be determined by measuring cleaved caspase 3 in endothelial lysates and Annexin-V binding by flow cytometry. In particular, endothelial activation may be determined by measuring P-selectin/ICAM-1 levels and NF KB nuclear localization by immunoblotting and immunofluorescence, respectively.

### Example 5—Murine E. coli Treatment Microbubble and miRNA

E. coli infection is the most common cause of ventilator-associated pneumonia. In vivo demonstration of the use of USMB in the treatment of E. coli infection in mice is described to assess the efficacy of antibiotic therapy for pneumonia-induced ARDS in the setting of ultrasound microbubble (USMB) therapy. Mouse models of ARDS are characterized by alveolar neutrophil recruitment, arterial hypoxemia and non-cardiogenic pulmonary edema. In the first, mice were inoculated intranasally with E. coli, and the animals' weight, activity score, oxygen saturation (by pulse oximetry) and body temperature were closely monitored. Colony forming units (cfu) from lung homogenates were measured to quantify bacterial growth.

### Example 6—Targeted Delivery Cf Antibiotics to Injured Regions Cf the Lung in E. coli Pneumonia

Mice infected with 1×10 7 cfu of E. coli (ATCC 25922) by intratracheal instillation are administered 1.5-6.0 mg/kg gentamicin by intraperitoneal injection 6 hours later. The dose of E. coli achieves significant lung injury, as shown in FIG. 13 panel C, with survival for at least 24 hours (to allow time for antibiotics to have an effect). This dose of gentamicin based on pilot studies is known to achieve incomplete sterilization of the lung. From a clinical standpoint, it is also desirable to use relatively low doses of aminoglycosides since they can cause serious renal and vestibulo-cochlear damage. A control subset of infected mice received no antibiotics. 1×10⁹ microbubbles was administered by tail vein followed by thoracic ultrasound using the same settings as in Example 1). A control subset of mice received microbubbles or ultrasound alone without antibiotic. Mice are sacrificed 18 hours later and lungs are homogenized and plated for bacterial colonies. Lungs can also be processed for histology and scored by a pathologist in a blinded fashion for histological evidence of lung injury. Monitoring of oxygen saturation, activity score, body temperature and weight loss provides additional information on systemic response to treatment. Murine renal function can also be monitored, for example using serum creatinine by ELISA.

Varying doses of E. coli and varying amounts of microbubbles can be dosed, as guided by the safety studies in Example 1. The amount of gentamicin deposited in the lung compared to non-pulmonary organs by ELISA (commercially available) of tissue homogenates to quantify the degree of lung enrichment by USMB. Sequential ELISAs for gentamicin on bronchoalveolar lavage fluid and mouse plasma can be used to quantify the effect of USMB therapy on the pharmacokinetics of the antibiotic under study. Similar experiments can be done using S. aureus (ATCC 29213) to induce gram-positive pneumonia, with infected mice receiving vancomycin with or without USMB.

### Example 7—Targeted Gene Delivery

Influenza-infected and control mice are injected intravenously with microbubbles tagged with a plasmid encoding green fluorescent protein (GFP-tagged microbubbles). The influenza model is used in this experiment as its longer timeframe allows more time for protein expression. A subset of infected mice receive GFP-tagged microbubbles alone with or without ultrasound. Mice are monitored for oxygen saturation and signs of respiratory distress during and after the experiment. 24-48 hours after injection, mice are sacrificed and lungs are collected and assessed for evidence of gene delivery (i.e. expression of green fluorescent protein in the tissue) and blinded histological examination of lung injury. In particular, the correlation or colocalization between the expression of GFP and the degree of lung injury (e.g. neutrophil infiltration, septal thickening [14)) is calculated. The heart, kidneys, liver and spleen are also analyzed for off-target gene expression. A time course and dose-response is performed to determine how long after USMB it remains possible to detect GFP expression, such as, for example, 1-7 days. Circulating and tissue miRNA126-3p levels can be quantified after RNA extraction by quantitative real time PCR (8). Levels can also be determined in the lungs, kidney, spleen and heart to determine the degree of enrichment in the lung after USMB.

The following clauses are offered as further description of the examples of the apparatus. Any one or more of the following clauses may be combinable with any another one or more of the following clauses and/or with any subsection or a portion or portions of any other clause and/or combination and permutation of clauses. Any one of the following clauses may stand on its own merit without having to be combined with any other clause or any portion of any other clause, etc.

CLAUSE 1A: An apparatus 100 comprising: an ultrasound signal generator 102 for generating ultrasonic signals 202; and an ultrasound transducer assembly 104 having an ultrasound transducer 106, the ultrasound transducer 106 communicatively connected to the ultrasound signal generator 102, and the ultrasound transducer 106 configured to transmit the ultrasonic signals 202 generated by the ultrasound signal generator 102; wherein the ultrasound transducer 106 is configured to direct targeted ultrasound signals 202 to a chest cavity, the chest cavity including a lung 200, without moving the ultrasound transducer assembly 104; and the ultrasonic signals 202 are configured to induce microbubbles 306, using cavitation, to deliver a pharmaceutical product 400 to injured lung tissue 204. CLAUSE 2A: The apparatus of any of the clauses, wherein the ultrasound transducer assembly 104 is configured to be placed on a human chest 500. CLAUSE 3A: The apparatus of any of the clauses, wherein the ultrasound transducer assembly 104 is configured to be placed endobronchially. CLAUSE 4A: The apparatus of any of the clauses, wherein the ultrasound transducer 106 is configured to direct targeted ultrasound signals 202 to induce microbubbles 306 only once microbubbles 306 are detected in injured lung tissue 204. CLAUSE 5A: The apparatus of any of the clauses, wherein the ultrasound transducer 106 is configured to move within the ultrasound transducer apparatus 104. CLAUSE 6A: A method for delivering a pharmaceutical product 400 to injured lung tissue 204 in-vivo comprising: introducing microbubbles 306 to an area proximate to the injured lung tissue 204, and directing an ultrasonic signal 202 to the injured lung tissue 204, the ultrasonic signal 202 configured to induce microbubbles 306, using cavitation, to deliver a pharmaceutical product 400 to the injured lung tissue 204. CLAUSE 7A: The method of any of the clauses of this paragraph further comprising: prior to directing an ultrasonic signal 202, scanning a chest cavity to identify internal structures including organs, injured lung tissue 204, and health lung tissue. CLAUSE 8A: The method of any of the clauses further comprising: once the ultrasonic signal 202 has been directed to the injured lung tissue 204 to induce microbubbles 306 to deliver a pharmaceutical product 400 to the injured lung tissue 204, scanning the affected area for changes in the affected area. CLAUSE 9A: The method of any of the clauses further comprising: prior to directing an ultrasonic signal 202, detecting whether microbubbles 306 are in the area proximate to the injured lung tissue 204. CLAUSE 10A: A method comprising: using an ultrasound-mediated microbubble 306 to deliver a pharmaceutical product 400 to injured lung tissue 204. CLAUSE 11A: A system comprising: an apparatus 100 for using an ultrasound-mediated microbubble 306 to deliver a pharmaceutical product 400 to injured lung tissue 204.

CLAUSE 1: A pulmonary ultrasound apparatus comprising: an ultrasound signal generator for generating an ultrasonic signal; an ultrasound transducer assembly having an ultrasound transducer operatively connected to the ultrasound signal generator, the ultrasound transducer configured to transmit the ultrasound signal generated by the ultrasound signal generator to pulmonary tissue; wherein the ultrasonic signal is transmitted at a frequency, a pressure, and a pulse duration for cavitating microbubbles to deliver a pharmaceutically active molecule to the pulmonary tissue. CLAUSE 2: The apparatus of any of the clauses, wherein the ultrasound transducer assembly has a flexible planar body for covering the at least part of the chest, and without movement of the ultrasound transducer assembly, the ultrasound transducer transmits the ultrasonic signal to the pulmonary tissue. CLAUSE 3: The apparatus of any of the clauses, wherein the ultrasound transducer has a width and a length about the size of the intercostal muscles for transmitting the ultrasonic signal between the ribs. CLAUSE 4: The apparatus of any of the clauses, wherein the ultrasound transducer assembly has an elongated body for endobronchial insertion, and the ultrasound transducer is at a distal end of the elongated body for transmitting the ultrasonic signal to the pulmonary tissue. CLAUSE 5: The apparatus of any of the clauses, wherein the ultrasound transducer directs targeted ultrasound signals to cavitate the microbubbles only when the microbubbles are detected in the pulmonary tissue. CLAUSE 6: The apparatus of any of the clauses, wherein the ultrasound transducer assembly has a movement mechanism for moving the ultrasound transducer assembly within the ultrasound transducer assembly. CLAUSE 7: The apparatus of any of the clauses, further comprising: an array of ultrasound transducers, the array of ultrasound transducer spaced apart a distance and each ultrasound transducer is operatively connected to the ultrasound signal generator. CLAUSE 8: The apparatus of any of the clauses, further comprising: a 2-dimensional array of ultrasound transducers, the array of ultrasound transducer spaced apart a first distance in a first dimension and a second distance in a second dimension, and each ultrasound transducer is operatively connected to the ultrasound signal generator. CLAUSE 9: The apparatus of any of the clauses, wherein: the ultrasound transducer or the array of ultrasound transducers is capable of imaging, treating, or both imaging and treating an entire lung or both lungs entirely. CLAUSE 10: The apparatus of any of the clauses, wherein: the ultrasound transducer assembly is configured to maintain an adjustable spacing between the ultrasound transducers. CLAUSE 11: An intravenous composition for treating pulmonary edema, the composition comprising: microbubbles; a pharmaceutically active molecule; and a pharmaceutically acceptable carrier. CLAUSE 12: The composition of any of the clauses, wherein the pharmaceutically active molecule is at least one of microRNA, antagomir, and blockmir CLAUSE 13:The composition of any of the clauses, wherein the microRNA is at least one of miRNA-126, miRNA-150, miRNA-181b, miRNA126-3p, and miRNA150-5p; the antagomir is at least one of an antagomir to miRNA-27A and miRNA-146b; and the blockmir is at least one of a blockmir to miRNA-27A and miRNA-146b. CLAUSE 14: The composition of any of the clauses, wherein the pharmaceutically active molecule is at least one of an aminoglycoside, steroid, antibiotic, vancomycin, antiviral, endothelial barrier-enhancing drug, and keratinocyte growth factor (KGF). CLAUSE 15: The composition of any of the clauses, wherein the endothelial barrier-enhancing drug is one or more of vasculotide, adrenomedullin, fasudil, imatinib, atrial natriuretic peptide, and SIP. CLAUSE 16: The composition of any of the clauses, comprising microRNA and at least one of an antibiotic compound and an antiviral compound. CLAUSE 17: The composition of any of the clauses, wherein the microbubble is coformulated with the pharmaceutically active compound. CLAUSE 18: The composition any of the clauses, wherein the microbubble is formulated independently and added to a solution of pharmaceutically active compound. CLAUSE 19: The composition of any of the clauses, wherein the microbubble is bound to the pharmaceutically active compound. CLAUSE 20: Use of pulmonary ultrasound to treat pulmonary edema comprising: providing an intravenous composition to a patient comprising a plurality of microbubbles, a pharmaceutically active compound, and a pharmaceutically acceptable carrier; and applying ultrasound to the patient at a target of pulmonary edema to cavitate the microbubbles and deliver the pharmaceutically active compound to the patient. CLAUSE 21: The use of any of the clauses, wherein the pharmaceutically active molecule is at least one of a microRNA, antagomir, and blockmir CLAUSE 22: The use of any of the clauses, wherein the microRNA is one or more of miRNA-126, miRNA-150, miRNA-181b, miRNA126-3p, and miRNA150-5p; the antagomir is at least one of an antagomir to miRNA-27A and miRNA-146b; and the blockmir is at least one of a blockmir to miRNA-27A and miRNA-146b. CLAUSE 23: The use of any of the clauses, wherein the pharmaceutically active molecule is at least one of an aminoglycoside, antibiotic, steroid, vancomycin, antiviral, endothelial barrier-enhancing drug, and keratinocyte growth factor (KGF). CLAUSE 24: The use of any of the clauses, wherein the endothelial barrier-enhancing drug is one or more of vasculotide, adrenomedullin, fasudil, imatinib, atrial natriuretic peptide, and SIP. CLAUSE 25: The use of any of the clauses, wherein the intravenous composition comprises microRNA and at least one of an antibiotic compound and an antiviral compound. CLAUSE 26: A method of treating pulmonary edema, the method comprising: administering intravenously to a patient: microbubbles; and a pharmaceutically active molecule; and irradiating the patient with ultrasound at a target of pulmonary edema to deliver the pharmaceutically active compound to the patient. CLAUSE 27: The method of any of the clauses, wherein the pulmonary edema is associated with acute respiratory distress syndrome. CLAUSE 28: The method of any of the clauses, wherein the pulmonary edema is associated with cystic fibrosis. CLAUSE 29. The method of any of the clauses, wherein the pulmonary edema is associated with congestive heart failure. CLAUSE 30: The method of any of the clauses, wherein the pharmaceutically active molecule is one or more of microRNA, an aminoglycoside, vancomycin, antiviral, endothelial barrier-enhancing drug, and keratinocyte growth factor (KGF). CLAUSE 31: The method of any of the clauses, wherein the microbubbles and the pharmaceutically active molecule are delivered to the patient simultaneously. CLAUSE 32: The method of any of the clauses, wherein the microbubbles and the pharmaceutically active molecule are delivered to the patient sequentially. CLAUSE 33: The method of any of the clauses, wherein the target of pulmonary edema is lung endothelium lining. CLAUSE 34. A method for delivering a pharmaceutical active molecule to a site of pulmonary edema, comprising: introducing microbubbles to an area proximate to the site of pulmonary edema; and directing an ultrasonic signal to the site of pulmonary edema, the ultrasonic signal for cavitating the microbubbles to deliver the pharmaceutically active molecule to the site of pulmonary edema. CLAUSE 35: The method of any of the clauses, further comprising: scanning a chest cavity to identify internal structures including one or more organs, site of pulmonary edema, injured lung tissue, and healthy lung tissue. CLAUSE 36: The method of any of the clauses, further comprising: scanning the site of pulmonary edema for echogenicity changes in the injured lung tissue after delivery of the pharmaceutically active molecule to the site of pulmonary edema. CLAUSE 37: The method of any of the clauses, further comprising: detecting whether microbubbles are in the area proximate to the site of pulmonary edema. CLAUSE 38: The method of any of the clauses, further comprising: directing an ultrasonic signal to the site of pulmonary edema, the ultrasonic signal for cavitating the microbubbles, only when the microbubbles are detected in the area proximate to the site of pulmonary edema.

This written description uses examples to disclose the invention, including the best mode, and also to enable any person skilled in the art to make and use the invention. The patentable scope of the invention is defined by the claims, and may include other examples that occur to those skilled in the art. Such other examples are within the scope of the claims if they have structural elements that do not differ from the literal language of the claims, or if they include equivalent structural elements with insubstantial differences from the literal language of the claims.

It may be appreciated that the assemblies and modules described above may be connected with each other as required to perform desired functions and tasks within the scope of persons of skill in the art to make such combinations and permutations without having to describe each and every one in explicit terms. There is no particular assembly or component that may be superior to any of the equivalents available to the person skilled in the art. There is no particular mode of practicing the disclosed subject matter that is superior to others, so long as the functions may be performed. It is believed that all the crucial aspects of the disclosed subject matter have been provided in this document. It is understood that the scope of the present invention is limited to the scope provided by the independent claim(s), and it is also understood that the scope of the present invention is not limited to: (i) the dependent claims, (ii) the detailed description of the non-limiting embodiments, (iii) the summary, (iv) the abstract, and/or (v) the description provided outside of this document (that is, outside of the instant application as filed, as prosecuted, and/or as granted). It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

What is claimed is:

1. A pulmonary ultrasound apparatus comprising:
an ultrasound signal generator for generating an ultrasonic signal;
an ultrasound transducer assembly having an ultrasound transducer operatively connected to the ultrasound signal generator, the ultrasound transducer configured to transmit the ultrasound signal generated by the ultrasound signal generator to pulmonary tissue;
wherein the ultrasonic signal is transmitted at a frequency, a pressure, and a pulse duration for cavitating microbubbles to deliver a pharmaceutically active molecule to the pulmonary tissue.

2. The apparatus of claim 1, wherein the ultrasound transducer assembly has a flexible planar body for covering the at least part of the chest, and without movement of the ultrasound transducer assembly, the ultrasound transducer transmits the ultrasonic signal to the pulmonary tissue.

3. The apparatus of claim 1 or 2, wherein the ultrasound transducer has a width and a length about the size of the intercostal muscles for transmitting the ultrasonic signal between the ribs.

4. The apparatus of claim 1 or 3, wherein the ultrasound transducer assembly has an elongated body for endobronchial insertion, and the ultrasound transducer is at a distal end of the elongated body for transmitting the ultrasonic signal to the pulmonary tissue.

5. The apparatus of any one of claims 1 to 4, wherein the ultrasound transducer directs targeted ultrasound signals to cavitate the microbubbles only when the microbubbles are detected in the pulmonary tissue.

6. The apparatus of any one of claims 1 to 5, wherein the ultrasound transducer assembly has a movement mechanism for moving the ultrasound transducer assembly within the ultrasound transducer assembly.

7. The apparatus of any one of claims 1 to 6, further comprising:
an array of ultrasound transducers, the array of ultrasound transducer spaced apart a distance and each ultrasound transducer is operatively connected to the ultrasound signal generator.

8. The apparatus of any one of claims 1 to 6, further comprising:
a 2-dimensional array of ultrasound transducers, the array of ultrasound transducer spaced apart a first distance in a first dimension and a second distance in a second dimension, and each ultrasound transducer is operatively connected to the ultrasound signal generator.

9. The apparatus of anyone of claims 1 to 8, wherein:
the ultrasound transducer or the array of ultrasound transducers is capable of imaging, treating, or both imaging and treating an entire lung or both lungs entirely.

10. The apparatus of claims any one of claims 1 to 9, wherein:
the ultrasound transducer assembly is configured to maintain an adjustable spacing between the ultrasound transducers.

11. An intravenous composition for treating pulmonary edema, the composition comprising:
microbubbles;
a pharmaceutically active molecule; and
a pharmaceutically acceptable carrier.

12. The composition of claim 11, wherein the pharmaceutically active molecule is at least one of microRNA, antagomir, and blockmir.

13. The composition of claim 12, wherein the microRNA is at least one of miRNA-126, miRNA-150, miRNA-181b, miRNA126-3p, and miRNA150-5p; the antagomir is at least one of an antagomir to miRNA-27A and miRNA-146b; and the blockmir is at least one of a blockmir to miRNA-27A and miRNA-146b.

14. The composition of claim 11, wherein the pharmaceutically active molecule is at least one of an aminoglycoside, steroid, antibiotic, vancomycin, antiviral, endothelial barrier-enhancing drug, and keratinocyte growth factor (KGF).

15. The composition of claim 14, wherein the endothelial barrier-enhancing drug is one or more of vasculotide, adrenomedullin, fasudil, imatinib, atrial natriuretic peptide, and SIP.

16. The composition of claim 11, comprising microRNA and at least one of an antibiotic compound and an antiviral compound.

17. The composition of any one of claims 11-16, wherein the microbubble is coformulated with the pharmaceutically active compound.

18. The composition of any one of claims 11-17, wherein the microbubble is formulated independently and added to a solution of pharmaceutically active compound.

19. The composition of any one of claims 11-18, wherein the microbubble is bound to the pharmaceutically active compound.

20. Use of pulmonary ultrasound to treat pulmonary edema comprising:
providing an intravenous composition to a patient comprising a plurality of microbubbles, a pharmaceutically active compound, and a pharmaceutically acceptable carrier; and
applying ultrasound to the patient at a target of pulmonary edema to cavitate the microbubbles and deliver the pharmaceutically active compound to the patient.

21. The use of claim 20, wherein the pharmaceutically active molecule is at least one of a microRNA, antagomir, and blockmir.

22. The use of claim 21, wherein the microRNA is one or more of miRNA-126, miRNA-150, miRNA-181b, miRNA126-3p, and miRNA150-5p; the antagomir is at least one of an antagomir to miRNA-27A and miRNA-146b; and the blockmir is at least one of a blockmir to miRNA-27A and miRNA-146b.

23. The use of claim 20, wherein the pharmaceutically active molecule is at least one of an aminoglycoside, antibiotic, steroid, vancomycin, antiviral, endothelial barrier-enhancing drug, and keratinocyte growth factor (KGF).

24. The use of claim 23, wherein the endothelial barrier-enhancing drug is one or more of vasculotide, adrenomedullin, fasudil, imatinib, atrial natriuretic peptide, and SIP.

25. The use of claim 20, wherein the intravenous composition comprises microRNA and at least one of an antibiotic compound and an antiviral compound.

26. A method of treating pulmonary edema, the method comprising:
administering intravenously to a patient:
microbubbles; and
a pharmaceutically active molecule; and
irradiating the patient with ultrasound at a target of pulmonary edema to deliver the pharmaceutically active compound to the patient.

27. The method of claim 26, wherein the pulmonary edema is associated with acute respiratory distress syndrome.

28. The method of claim 26, wherein the pulmonary edema is associated with cystic fibrosis.

29. The method of claim 26, wherein the pulmonary edema is associated with congestive heart failure.

30. The method of any one of claims 26-29, wherein the pharmaceutically active molecule is one or more of microRNA, an aminoglycoside, vancomycin, antiviral, endothelial barrier-enhancing drug, and keratinocyte growth factor (KGF).

31. The method of any one of claims 26-30, wherein the microbubbles and the pharmaceutically active molecule are delivered to the patient simultaneously.

32. The method of any one of claims 26-30, wherein the microbubbles and the pharmaceutically active molecule are delivered to the patient sequentially.

33. The method of any one of claims 26-32, wherein the target of pulmonary edema is lung endothelium lining.

34. A method for delivering a pharmaceutical active molecule to a site of pulmonary edema, comprising:
introducing microbubbles to an area proximate to the site of pulmonary edema; and
directing an ultrasonic signal to the site of pulmonary edema, the ultrasonic signal for cavitating the microbubbles to deliver the pharmaceutically active molecule to the site of pulmonary edema.

35. The method of claim 34, further comprising:
scanning a chest cavity to identify internal structures including one or more organs, site of pulmonary edema, injured lung tissue, and healthy lung tissue.

36. The method of claim 34 or 35, further comprising:
scanning the site of pulmonary edema for echogenicity changes in the injured lung tissue after delivery of the pharmaceutically active molecule to the site of pulmonary edema.

37. The method of any one of claims 34 to 36, further comprising:
detecting whether microbubbles are in the area proximate to the site of pulmonary edema.

38. The method of any one of claims 34 to 37, further comprising:
directing an ultrasonic signal to the site of pulmonary edema, the ultrasonic signal for cavitating the microbubbles, only when the microbubbles are detected in the area proximate to the site of pulmonary edema.
